SEQUENCE LISTING (28 sequences)

SEQ ID NO: 1 - protein, 7 aa, synthetic construct
PLGLFAR

SEQ ID NO: 2 - protein, 5 aa, synthetic construct
PLGVR

SEQ ID NO: 3 - protein, 4 aa, synthetic construct
PLAL

SEQ ID NO: 4 - protein, 20 aa, synthetic construct
  VARIANT 1: note = X is I, T, V or nVa
  VARIANT 3: note = X is I, T, V or nVa
  VARIANT 5: note = X is I, T, V or nVa
  VARIANT 18: note = X is I, T, V or nVa
  VARIANT 20: note = X is I, T, V or nVa
XKXEXKVKVPPTELRTKXKX

SEQ ID NO: 5 - protein, 22 aa, synthetic construct
  VARIANT 1: note = X is I, T, V or nVa
  VARIANT 3: note = X is I, T, V or nVa
  VARIANT 5: note = X is I, T, V or nVa
  VARIANT 20: note = X is I, T, V or nVa
  VARIANT 22: note = X is I, T, V or nVa
XKXEXKVKVPPLGLFARTKXKX

SEQ ID NO: 6 - protein, 20 aa, synthetic construct
  VARIANT 1: note = X is I, T, V or nVa
  VARIANT 3: note = X is I, T, V or nVa
  VARIANT 5: note = X is I, T, V or nVa
  VARIANT 18: note = X is I, T, V or nVa
  VARIANT 20: note = X is I, T, V or nVa
XKXEXKVKVPPLGVRTKXKX

SEQ ID NO: 7 - protein, 20 aa, synthetic construct
IKTEIKVKVPPLGVRTKIKV

SEQ ID NO: 8 - protein, 20 aa, synthetic construct
IKVEIKVKVPPLGVRTKIKV

SEQ ID NO: 9 - protein, 20 aa, synthetic construct
  MOD_RES 1: note = Nva
  MOD_RES 5: note = Nva
  MOD_RES 18: note = Nva
  MOD_RES 20: note = Nva
VKTEVKVKVPPLGVRTKVKV

SEQ ID NO: 10 - protein, 20 aa, synthetic construct
  MOD_RES 1: note = Nva
  MOD_RES 3: note = Nva
  MOD_RES 5: note = Nva
  MOD_RES 18: note = Nva
  MOD_RES 20: note = Nva
VKVEVKVKVPPLGVRTKVKV

SEQ ID NO: 11 - protein, 11 aa, synthetic construct
IKVEIKVKVPP

SEQ ID NO: 12 - protein, 20 aa, synthetic construct
IKVEIKVKVPPTKIAVLTAV

SEQ ID NO: 13 - protein, 20 aa, synthetic construct
IKVEIKVKVPPLALETKIKV

SEQ ID NO: 14 - protein, 20 aa, synthetic construct
IKVEIKVKVPPVSLLTAIKV

SEQ ID NO: 15 - protein, 12 aa, synthetic construct
  VARIANT 8: note = X is T or V
  VARIANT 11: note = X is K or E
VPPTEIKXKIXV

SEQ ID NO: 16 - protein, 12 aa, synthetic construct
  VARIANT 8: note = X is T or V
  VARIANT 11: note = X is K or E
VPPTKIKXKIXV

SEQ ID NO: 17 - protein, 12 aa, synthetic construct
  VARIANT 8: note = X is T or V
  VARIANT 11: note = X is K or E
VPPTELRXKIXV

SEQ ID NO: 18 - protein, 20 aa, synthetic construct
IKIAVLVKVPPTEIKTKIKV

SEQ ID NO: 19 - protein, 20 aa, synthetic construct
IKVSLLTAVPPTKIKVKIEV

SEQ ID NO: 20 - protein, 20 aa, synthetic construct
IKVKIELRVPPTELRTKIKV

SEQ ID NO: 21 - protein, 20 aa, synthetic construct
VKVKVKVKVPPTKVEVKVKV

SEQ ID NO: 22 - protein, 20 aa, synthetic construct
VLTKVKTKVPPTKVEVKVLV

SEQ ID NO: 23 - protein, 20 aa, synthetic construct
IKVKIKVKVPPTELRVKIKV

SEQ ID NO: 24 - protein, 20 aa, synthetic construct
IKVKIELRVPPTKIKVKIKV

SEQ ID NO: 25 - protein, 20 aa, synthetic construct
VKVKVKVKVPPLGLFARVKV

SEQ ID NO: 26 - protein, 20 aa, synthetic construct
VKIKVKIKVPPLGLFARIKV

SEQ ID NO: 27 - protein, 20 aa, synthetic construct
IKVKIKVKVPPLGVRVKIKV

SEQ ID NO: 28 - protein, 20 aa, synthetic construct
  MOD_RES 1: note = Nva
  MOD_RES 3: note = Nva
  MOD_RES 5: note = Nva
  MOD_RES 16: note = Nva
  MOD_RES 18: note = Nva
  MOD_RES 20: note = Nva
VKVKVKVKVPPLGVRVKVKV

(19) United States
(12) Patent Application Publication
(10) Pub. No.: US 2026/0069536 A1
(43) Pub. Date: Mar. 12, 2026

(54) COMPOSITIONS, SYSTEMS, AND METHODS FOR DELIVERY OF THERAPEUTICS

(71) Applicants: THE JOHNS HOPKINS UNIVERSITY, Baltimore, MD (US); THE UNITED STATES OF AMERICA, AS REPRESENTED BY THE SECRETARY,, Bethesda, MD (US)

(72) Inventors: Giorgio Raimondi, Baltimore, MD (US); Joel P. Schneider, Bethesda, MD (US); Julia Patrone, Baltimore, MD (US); Alexander Komin, Baltimore, MD (US); Monessha Nambiar, Bethesda, MD (US); Xiomara Calderon-Colon, Baltimore, MD (US); Olivia Tiburzi, Baltimore, MD (US)

(21) Appl. No.: 18/872,676

(22) PCT Filed: Jun. 8, 2023
(86) PCT No.: PCT/US2023/068139

§ 371 Date: Dec. 6, 2024

Related U.S. Application Data
(60) Provisional application No. 63/350,315, filed on Jun. 8, 2022.

(51) Int. Cl. A61K 9/06; A61K 9/51; A61K 31/519; A61K 38/17; A61K 47/42; A61P 37/06

(52) U.S. Cl. CPC A61K 9/06; A61K 9/5123; A61K 31/519; A61K 38/1774; A61K 47/42; A61P 37/06

(57) ABSTRACT

The present disclosure provides compositions, systems, devices, and methods for the delivery of therapeutics. Particularly, the disclosure provides composition, systems, and devices of the delivery of Janus kinase (JAK) inhibitors and uses thereof, such as for inhibiting allograft rejection or treating autoimmune diseases.

### CROSS-REFERENCE TO RELATED APPLICATIONS

This application claims the benefit of U.S. Provisional Application No. 63/350,315, filed Jun. 8, 2022, the content of which is herein incorporated by reference in its entirety.

### STATEMENT REGARDING FEDERALLY-SPONSORED RESEARCH OR DEVELOPMENT

This invention was made with Government support under grant numbers W81XWH-18-1-0789, W81XWH-18-1-0792 and W81XWH-18-1-0793, awarded by the Army Medical Research and Development Command. The Government has certain rights in the invention.

### FIELD

The present disclosure provides compositions, systems, devices, and methods for the delivery of therapeutics. Particularly, the disclosure provides composition, systems, and devices of the delivery of Janus kinase (JAK) inhibitors and uses thereof.

### SEQUENCE LISTING STATEMENT

The contents of the electronic sequence listing titled JHU-40817-252.xml (Size: 38,787 bytes; and Date of Creation: Oct. 20, 2025) is herein incorporated by reference in its entirety.

### BACKGROUND

Transplantation remains the only therapeutic option for end-stage organ diseases and an option after devastating tissue loss. However, the debilitating side effects (e.g., nephrotoxicity, cardiovascular disease, diabetes, cancer) of ensuing chronic high-dose multi drug immunosuppressive therapy, together with still inadequate control of alloimmunity, counterbalances its benefits and prevents its widespread use. Costimulation blockade, via the biologic CTLA4-Ig, has shown promising improvements for immune modulation following solid organ transplantation. However, these benefits are offset by unacceptably high rates of acute rejection episodes

### SUMMARY

Disclosed herein are hydrogel compositions comprising a first JAK inhibitor, or a pharmaceutically acceptable salt thereof, and a second JAK inhibitor, or a pharmaceutically acceptable salt thereof, wherein the second JAK inhibitor is encapsulated in a lipid nanoparticle. In some embodiments, the first JAK inhibitor, or a pharmaceutically acceptable salt thereof, and the second JAK inhibitor, or a pharmaceutically acceptable salt thereof, are different compounds. In some embodiments, the first JAK inhibitor, or a pharmaceutically acceptable salt thereof, and the second JAK inhibitor, or a pharmaceutically acceptable salt thereof, are the same compounds. In some embodiments, the first JAK inhibitor, the second JAK inhibitor, or both is tofacitinib, or a pharmaceutically acceptable salt thereof.

In some embodiments, the first JAK inhibitor, or a pharmaceutically acceptable salt thereof, is contained within microcrystalline deposits.

In some embodiments, the lipid nanoparticle comprises a lipid core comprising a saturated hydrocarbon having 19 to 30 carbons and a liquid lipid.

In some embodiments, the lipid core is surrounded by a surfactant layer comprising at least one non-ionic surfactant. In some embodiments, the at least one non-ionic surfactant comprises a polyethylene glycol glyceride, a PEG ester, or a combination thereof. In some embodiments, the surfactant layer further comprises an anionic surfactant. In some embodiments, the anionic surfactant comprises 10-15% of the surfactant layer. In some embodiments, the anionic surfactant is a glycolic acid ethoxylate ether.

In some embodiments, the lipid nanoparticle has a lipid to surfactant ratio of 1:0.5 to 1:2.

In some embodiments, the lipid nanoparticle further comprises a polymer.

In some embodiments, the hydrogel is a peptide hydrogel.

In some embodiments, the hydrogel is protease sensitive. In some embodiments, the hydrogel comprises peptides having matrix metallo-proteases (MMP) cleavage sites. In some embodiments, the MMP cleavage site has an amino acid sequence selected from the group consisting of ELR, PLGLFAR (SEQ ID NO: 1), PLGVR (SEQ ID NO: 2), X₁X₂X₃X₄, X₅SX₆LX₇A, and PLAL (SEQ ID NO: 3), wherein X₁ is L or I, X₂, and X₃ are independently selected from any amino acid, X₄ is a hydrophobic amino acid, X₅ is a hydrophobic amino acid, X₆ is any amino acid, and X₇ is T or L.

In some embodiments, the peptides comprise an amino acid sequence of: Z₁KZ₂EZ₃KVKVPPTELRTKZ₄KZ₅ (SEQ ID NO: 4); Z₁KZ₂EZ₃KVKVPPLGLFARTKZ₄KZ₅ (SEQ ID NO: 5); or Z₁KZ₂EZ₃KVKVPPLGVRTKZ₄KZ₅ (SEQ ID NO: 6), wherein each of Z₁, Z₂, Z₃, Z₄, and Z₅ are independently selected from I, T, V, and norvaline. In some embodiments, the peptides comprise an amino acid sequence selected from: IKTEIKVKV^{D}PPLGVRTKIKV (SEQ ID NO: 7); IKVEIKVKV^{D}PPLGVRTKIKV (SEQ ID NO: 8); (nV)KTE(nV)KVKV^{D}PPLGVRTK(nV)K(nV) (SEQ ID NO: 9); and (nV)K(nV)E(nV)KVKV^{D}PPLGVRTK(nV)K(nV) (SEQ ID NO: 10), wherein nV is norvaline.

In some embodiments, the peptides comprise an amino acid sequence of IKVEIKVKV^{D}PP (SEQ ID NO: 11)-Z₆-V, wherein Z₆ is a 5-10 amino acid sequence comprising the MMP cleavage site. In some embodiments, the peptides comprise an amino acid sequence selected from:

(SEQ ID NO: 12)
IKVEIKVKV^{D}PPTKIAVLTAV;
(SEQ ID NO: 13)
IKVEIKVKV^{D}PPLALETKIKV;
and
(SEQ ID NO: 14)
IKVEIKVKV^{D}PPVSLLTAIKV.

In some embodiments, the peptides comprise an amino acid sequence of IK-Z₇-V^{D}PPTEIKZ₈KIZ₉V (SEQ ID NO: 15); IK-Z₇-V^{D}PPTKIKZ₈KIZ₉V (SEQ ID NO: 16); or IK-Z₇-V^{D}PPTELRZ₈KIZ₉V (SEQ ID NO: 17), wherein Z₇ is a 5-10 amino acid sequence comprising the MMP cleavage site, Z₈ is T or V, and Z₉ is K or E. In some embodiments, the peptides comprise an amino acid sequence selected from: IKIAVLVKV^{D}PPTEIKTKIKV (SEQ ID NO: 18); IKVSLLTAV^{D}PPTKIKVKIEV (SEQ ID NO: 19); and IKVKIELRV^{D}PPTELRTKIKV (SEQ ID NO: 20).

In some embodiments, the hydrogel is a shear-thinning hydrogel.

In some embodiments, the hydrogel further comprises a pharmaceutically acceptable carrier.

Also provided herein is a pre-filled delivery device comprising a hydrogel composition as disclosed herein. In some embodiments, the device is a syringe. In some embodiments, the device comprises a single dose of the hydrogel composition.

Further provided are methods of using the hydrogel compositions disclosed herein for administration to a subject. In some embodiments, the methods further comprise administering cytotoxic T-lymphocyte-associated protein 4 (CTLA4)-Ig to the subject.

In some embodiments, the methods include inhibiting transplant rejection in a subject who has received an organ or tissue transplant, comprising administering a hydrogel composition at or adjacent to the site of the transplant. In some embodiments, the transplant is an allograft. In some embodiments, the allograft is a solid organ. In some embodiments, the allograft is a vascularized composite allograft (VCA). In some embodiments, the hydrogel composition is contained within the VCA.

In some embodiments, the methods comprise treating or preventing a disease or disorder. In some embodiments, the disease or disorder is an autoimmune or an inflammatory disease or disorder.

Other aspects and embodiments of the disclosure will be apparent in light of the following detailed description and accompanying figures.

### BRIEF DESCRIPTION OF THE DRAWINGS

FIG. 1 is a schematic of a proposed dual component delivery platform as described herein.

FIG. 2 is a graph of the survival curves of B6 to BALB/c heart transplants with indicated treatments showing that an enhanced co-stimulation blockade promotes survival and protects ischemic grafts. n=4-5 for each group. IS indicates that donor hearts were kept ischemic for 4h. *P<0.05.

FIG. 3A is a schematic of localized delivery of Tofacitinib (Tofa) microcrystals from a hydrogel (MTH) implanted in mice receiving heterotopic heart transplant. FIG. 3B is survival curves of C57Bl/6 mice receiving BALB/c heart transplants under the indicated treatments.

FIGS. 4A-4D show Tofa-lipid nanoparticles (LNp) inhibit DC maturation and function. FIGS. 4A and 4B show representative uptake of fluorescent LNp by mouse DCs measured via flow cytometry at the indicated time point (FIG. 4A) and confocal imaging (CD11c+: cyan; LNp: red) at 30 min (FIG. 4B). FIG. 4C shows maturation marker expression on DCs left untreated (grey), stimulated with LPS (blue), or pre-incubated with Tofa-LNp before LPS (green). FIG. 4D shows representative proliferation of allogeneic T cells stimulated by LPS-matured DC that were unmodified (left column) or pre-conditioned with Tofa-LNp (right column). Numbers indicate percentage of proliferating cells.

FIGS. 5A-5C show the in vivo distribution of LNp and impact on antigen presenting cells (APCs). FIGS. 5A and 5B are exemplary images of fluorescent LNp administered subcutaneously (FIG. 5A) or via oral gavage (FIG. 5B). Msn: mesenteric. Pnc: pancreatic. Ing: inguinal lymph nodes. Spl: spleen. FIG. 5C is a graph of the cumulative results of CD80 expression on CD11c^{hi}CD11b^{int} cell from mice receiving Tofa-LNp via oral gavage then challenged with LPS.

FIGS. 6A-6C show LNp encapsulation in a hydrogel. FIG. 6A is a graph of the comparative storage moduli (G′) of MAX8 gels with and without Neut-NLCs and Ani2-NLCs. FIG. 6B is a graph of the cumulative release profiles of Neut-NLC and Ani2-NLC from MAX8 gels. FIG. 6C is an image of LNp retention by MAX8 gels. A lipophilic dye (pink) was encapsulated within the NLCs to quantify release after 5 days.

FIG. 7 is a schematic of an exemplary method for evaluation of short-term and long-term effect of JAKi delivery on alloreactive T cells.

FIG. 8 is a graph of the in vitro drug release profiles of Tofa-LNp and Upa-LNp.

FIG. 9A-9C show MMP activity in vascularized composite allograft (VCA) rejection. FIG. 9A are images showing MMP activity in hind limb transplant recipients measured via IVIS at the indicated time. FIG. 9B is a graph of the cumulative results of MMP activity normalized to non-transplanted tissue. FIG. 9C is 3D renderings of MMP signal measured on POD7 in recipients of allogeneic (left) and syngeneic (right) limb.

FIGS. 10A and 10B show protease responsive LNp release. FIG. 10A is HPLC traces demonstrating proteolytic digestion of Peptide-4 gels. FIG. 10B is a graph of the release profiles of Ani2-NLC from Peptide-4 gels in response to trypsin.

FIG. 11 is a table of MMP-cleavable peptide sequences.

FIG. 12 shows Tofa-NLC mediated inhibition of cytokine signaling in B cells ex vivo. Mouse splenocytes were incubated for 2, 4, or 6 hours with Tofa-NLC that had been cleaned of free Tofa via desalting spin columns. Incubations were performed using two starting “concentration equivalents” of Tofa (as explained in the text): a low dose (Upper Panels; where the initial amount of encapsulated Tofa was 0.18 μM) and a high dose (Lower Panels; where the initial amount of encapsulated Tofa was 0.54 μM). At the of the respective incubation, IFN-β (100U/ml) was used to stimulate the cells and the induction of P-STAT1 in cell subsets was assessed via P-Flow. The two graphs in each row are different representations of the same populations/time points. Results are shown for gated B cells. Blue vertical lines mark the peak of P-STAT1 by IFN-β.

FIG. 13 shows Tofa-NLC impact on B cells at short pre-incubation times. Same experiment as in FIG. 12, but implementing shorter pre-incubations with cleaned Tofa-NLC: 20 min, 40 min, 1 h, and 2h. Data shown is on gated B cells for the 20 and 40 min pre-incubations.

FIG. 14 shows Ani2-LNp exhibit low cellular toxicity. Mouse bone marrow derived dendritic cells were co-cultured with increasing concentrations (as indicated above) of Ani2-LNp and incubated for 18 hours. At the end of the culture, cells were collected and stained for CD11c (to identify dendritic cells) and with a fixable viability dye (y axis). Numbers in graphs indicate the percentage of dying cells. Representative of two independent experiments. Cumulative data from triplicates of each culture conditions is presented to the right, where cell viability is expressed as change relative to the “Non-treated” condition.

FIG. 15 shows the confirmation of absence of cellular toxicity of Ani2 LNp at low concentration range. Representative results of the assessment of toxicity of Ani2 LNp performed as described in FIG. 14 exploring a range of concentrations below 100 mg/ml. Data is representative of triplicates with similar values.

FIG. 16 shows peptide sequences of MMP-cleavable hydrogels and a graph of the cumulative release profiles of Ani2 and Neut-NLCs from IVET1 and nVET2 gels. IVET2—SEQ ID NO: 7; IVET1—SEQ ID NO: 8; nVET2—SEQ ID NO: 9; nVET1—SEQ ID NO: 10.

FIG. 17 shows the effect of MMP12 on the native strength of IVET1 gels (n=3) over time.

FIGS. 18A-18C shows the cumulative release of 16 mM microcrystalline Tofa from 1 wt % gels of peptides 1, 2 and 3 (FIG. 18A); release of 16 mM and 32 mM microcrystalline Tofa from 1 wt % gels of nVET1 and IVET1 peptides (FIG. 18B); and comparative release rates of Ani2-NLCs from 1 wt % gels of nVET1 and IVET1 peptides in the presence and absence of enzymes, trypsin and MMP12 (FIG. 18C).

FIG. 19 shows the NLC distribution following subcutaneous injection. Representative uptake of HLDI-NLC by immune cells (T and B cells) of spleen (Spl), inguinal lymph nodes (iLN), and mesenteric lymph nodes (mLN) at 1 (blue, middle) or 2 hour (orange, right) following subcutaneous injection near the base of the tail. Cell populations and particle fluorescence were assessed via Spectral Flow Cytometry.

FIG. 20 shows the biocompatibility of hydrogel formulation. Representative sequential photos of site of injection (SQ) of 50 ul of Tofa-hydrogel in skin flap used to simulate the stress conditions of the tissue in the hind limb transplant model. No sign of toxicity caused by the hydrogel is apparent.

FIGS. 21A-21C show that single injection of Tofa-Hydrogel in VCA provides a significant, although limited, transplant survival. FIG. 21A is the mouse hind limb transplant model. FIG. 21B shows representation of localized eCoB regimen in mice receiving hind limb transplantation. FIG. 21C is the transplant survival curves in recipients subjected to the indicated treatment.

FIGS. 22A and 22B show that short-term and graft-restricted administration of Tofa-LNp promotes sustained regulation of VCA rejection. FIG. 22A is a representation of localized LNp-mediated eCoB regimen in mice receiving hind limb transplantation. FIG. 22B is transplant survival curves in recipients subjected to the indicated treatments.

### DETAILED DESCRIPTION

The present disclosure provides compositions and devices for local, multi-modal delivery of therapeutics, e.g., JAK inhibitors, and use in methods of treatment and prevention, e.g., for preventing transplant rejection, particularly in combination with systemic immunotherapies.

Except for very rare cases, T lymphocytes are at the core of transplant rejection: they control both the cellular and humoral arms of the rejection response. Costimulation blockade (CoB) therapies aim at regulating T cell activation by targeting necessary costimulatory receptors (like CD28). Belatacept, a second generation of the biologic CTLA4-Ig that sequesters the CD28 ligands CD80 and CD86, showed promise in primate studies and has been recently approved for maintenance therapy in kidney recipients. However, the use of belatacept as an immunosuppressive agent is characterized by a higher incidence of acute rejection episodes than conventional calcineurin inhibitor (CNI)-based protocols. A growing body of evidence shows that induction of long term survival by CoB is impaired by inflammatory responses, where the activation of alloreactive T cells can happen in a CD28-independent manner.

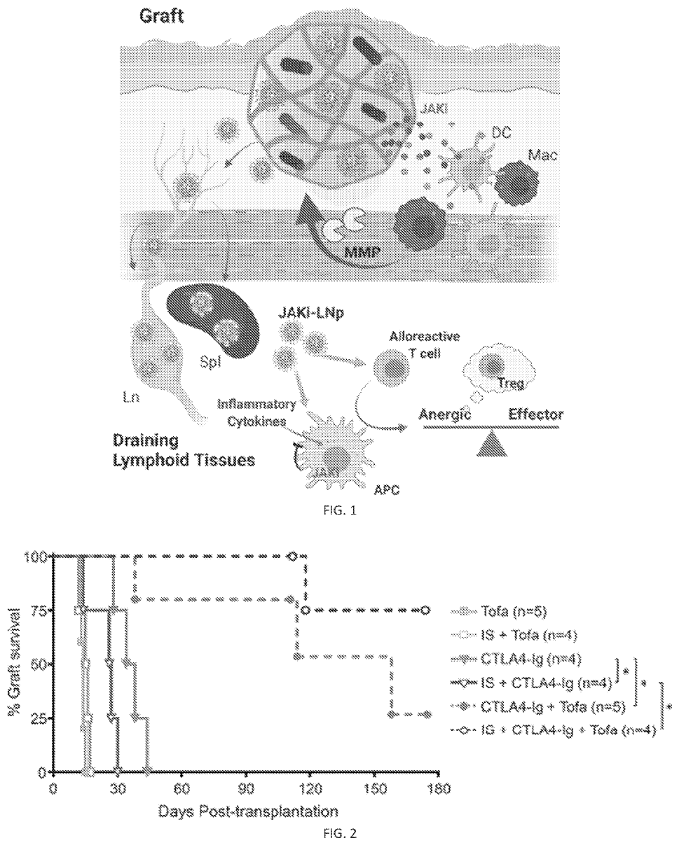

Pharmacological inhibition of inflammatory cytokines is particularly effective in synergizing with CTLA4-Ig to control the activation of alloreactive T cells and prevent rejection. Tofacitinib (Tofa), a Jak3/1 inhibitor that blocks signaling of a class of inflammatory cytokines, synergized with CTLA4-Ig to promote long-term transplant survival in a mouse model of heart transplantation. Tofa can be used as part of a short term treatment to potentiate the effect of CTLA4-Ig. This strategy was also effective when transplants are kept ischemic for many hours, a condition where CTLA4-Ig loses most of its therapeutic activity. The combination of Tofa+CTLA4-Ig is referred to herein as Enhanced Costimulation Blockade (ECoB). The in vivo use of JAK inhibitors (JAKi), e.g., Tofa, is affected by two limitations: 1) absorption and short half-life; and 2) deleterious side effects associated with high serum concentrations induced by systemic and prolonged administration. When combined with CTLA4-Ig, both innate and adaptive immune cells that infiltrate the graft and lymphocyte priming are modulated (FIG. 1). Differently from the proposed use of Tofa as a chronic immunosuppressant, data indicate that Tofa can be used as part of a short term treatment to potentiate the effect of CTLA4-Ig. This strategy is also effective when transplants are kept ischemic for many hours, a condition where CTLA4-Ig loses most of its therapeutic activity. Tofa has also an effect on NK, and B cells which may translate into reduced incidence of antibody mediated rejection. Combined with the observation that fewer patients on belatacept develop de novo donor specific antibodies, the combination of JAKi and Belatacept offers a potential safe and effective treatment for (transplanted) patients.

Provided herein are systems and compositions comprising a single injectable hydrogel material, referred to herein as [cJAKi-Hydro(JAKi-LNp)], containing both microcrystalline JAKi deposits and lipid nanoparticles encapsulating a JAKi. The compositions and systems facilitate creation of a sustained gradient of JAKi near the desired site while also releasing JAKi-containing lipid nanoparticles (LNp) that can condition immune cells in graft draining lymphoid tissues. JAKi is released locally to tissues with immediate sustained diffusion from the hydrogel followed by release of JAKi-LNp in response to localized enzymatic activity that is driven by the intensity of the rejection response.

These compositions and systems stabilize JAKi while providing a localized and targeted release, in some embodiments, tuned to the intensity of the rejection response. Additionally, these compositions and systems reduce the number of needed administrations, maximize the synergism of JAKi with CTLA4-Ig when and where needed, and minimize JAKi-associated side effects. In cases of transplants or grafts, JAKi-LNp can facilitate delivery of JAKi selectively to immune cells in the lymph nodes draining the graft, to an extent not achievable with free drug. This will render a regulated and localized synergism with systemic CTLA4-Ig (actuating ECoB). With the addition of a recent warning from the FDA on the chronic use of JAKi and the risk of cardiovascular events, the compositions and methods here provide a pathway to fully explore the potential and clinical translatability of JAKi and CoB/ECoB therapies without the toxicity and unwanted side effects so often observed with JAKi use.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “allograft” herein refers to the transplant of an organ, tissue, or cells from a donor to a recipient who is genetically non-identical to the donor. A variety of cells, tissues and organs can be used for allografts, including, but not limited to, heart, lung, liver, kidney, pancreas, intestine, skin, bone, ligament, tendon, cornea, face, limbs, islet cells, and bone marrow.

The term “hydrogel” herein refers to a specific type of gel in which water-swellable polymeric matrices that can absorb a substantial amount of water in a three-dimensional network of macromolecules held together by covalent or noncovalent crosslinks.

As used herein, the terms “effective amount” or “therapeutically effective amount,” refer to a sufficient amount of the compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of a composition as described herein required to provide a clinically significant decrease in disease symptoms.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide,” “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), norvaline (found in the antifungal peptide of Bacillus subtilis), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine(Cl) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, omithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, the terms “providing,” “administering,” and “introducing,” are used interchangeably herein and refer to the placement of the compositions of the disclosure into a subject by a method or route which results in at least partial localization to a desired site. The compositions can be administered by any appropriate route which results in delivery to a desired location in the subject.

As used herein, “treat,” “treating,” and the like means a slowing, stopping, or reversing of progression of an infection, disease, disorder and/or condition when provided a composition described herein to an appropriate subject. The term also includes a reversing of the progression of such an infection, disease, disorder and/or condition to a point of eliminating or greatly reducing the disease. As such, “treating” means an application the compositions described herein to a subject, where the subject has a disease, disorder and/or condition or a symptom of a disease, disorder and/or condition, where the purpose is to cure, heal, alleviate, relieve, alter, remedy, ameliorate, improve, or affect the disease, disorder and/or condition or symptoms of the disease, disorder and/or condition.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

A “syringe” is a device comprising a barrel, typically but not necessarily tube-shaped, for injecting/applying or withdrawing a sample in a thin stream, typically through a hollow needle. Samples are injected/applied or withdrawn via pressure, typically from a reciprocating pump (e.g., employing a piston or plunger). A plunger can be linearly pulled and pushed along the inside of the barrel, allowing the syringe to take in and expel liquid or gas through a discharge orifice at the front (open) end of the tube. The open end of the syringe may be fitted with a needle, a nozzle, or a tubing to help direct the flow into and out of the barrel.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. COMPOSITIONS

Disclosed herein are hydrogel compositions comprising a first JAK inhibitor and a second JAK inhibitor. In some embodiments, the first JAK inhibitor is in crystalline form. In some embodiments, the first JAK inhibitor is contained within microcrystalline deposits or aggregates. In some embodiments, the second JAK inhibitor is encapsulated in a lipid nanoparticle. Also disclosed herein are hydrogel compositions comprising a JAK inhibitor is encapsulated in a lipid nanoparticle. The compositions may be suitable for administration to a subject, which may be human or non-human.

In some embodiments, the hydrogel compositions comprise about 5 mM to about 35 mM first JAK inhibitor. For example, the compositions about 5 mM, about 10 mM, about 15 mM, about 20 mM, about 25 mM, about 30 mM, or about 35 mM of the first JAK inhibitor.

The JAK family plays a role in the cytokine-dependent regulation of proliferation and function of cells involved in immune response. Inhibitors of members of the JAK family have therapeutic efficacy in the treatment of cancer, and autoimmune and inflammatory diseases Currently, there are four known mammalian JAK family members: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2).

The JAK inhibitor may inhibit one or more of the JAK family members. In some embodiments, the JAK inhibitor decreases the kinase activity of JAKi. In some embodiments, the JAK inhibitor decreases the kinase activity of JAK2. In some embodiments, the JAK inhibitor decreases the kinase activity of JAK3. In some embodiments, the JAK inhibitor decreases the kinase activity of TYK2. In some embodiments, the JAK inhibitor decreases the kinase activity of JAK1 and JAK2. In some embodiments, the JAK inhibitor decreases the kinase activity of JAK1 and JAK3. In some embodiments, the JAK inhibitor decreases the kinase activity of JAK2 and JAK3. In some embodiments, the JAK inhibitor decreases the kinase activity of JAKI, JAK2, and JAK3. In some embodiments, the JAK inhibitor is a pan-JAK inhibitor.

Exemplary JAK inhibitors include, but are not limited to, to abrocitinib, baricitinib, cerdulatinib, delgocitinib, decernotinib, deucravacitinib, fedratinib, filgotinib, gandotinib, lestaurtinib, momelotinib, oclacitinib, parcitinib, peficitinib, ruxolitinib, solcitinib, tofacitinib, or upadacitinib. JAK inhibitor, as used herein, encompasses pharmaceutically acceptable salts and derivatives of a JAK inhibitor.

The JAK inhibitor may be in the form of a pharmaceutically acceptable salt. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

The first JAK inhibitor and the second JAK inhibitor may be different compounds. For example, the first JAK inhibitor may be upadacitinib whereas the second JAK inhibitor may be tofacitinib. Alternatively, the first JAK inhibitor and the second JAK inhibitor may be the same compounds. For example, in some embodiments, the first JAK inhibitor and the second JAK inhibitor are tofacitinib. Tofacitinib (3-((3R,4R)-4-methyl-3-(methyl(7H-pyrrolo[2,3-d]pyrimidin-4-yl)amino)piperidin-1-yl)-3-oxopropanenitrile), pharmaceutically acceptable salts and derivatives thereof are known (see, for example, PCT Publication Nos. WO 01/42246, WO 01/42246, WO 03/048162, WO 2012/135338, WO 2012/137111, and WO 2013/090490; and U.S. Pat. Nos. 9,260,438 and 9,670,160, which are herein incorporated by reference in their entirety).

### a) Lipid Nanoparticle

Lipid nanoparticles (LNp) suitable for use herein include solid lipid nanoparticles (SLN) or nanostructured lipid carriers (NLC). In some embodiments, the lipid nanoparticle is a nanostructured lipid carriers (NLC).

The lipid nanoparticle may comprise both solid and liquid lipid components. In some embodiments, the lipid nanoparticle comprises a lipid core comprising a saturated hydrocarbon having 19 to 30 carbons. Exemplary saturated hydrocarbons include, but are not limited to, nonadecane, eicosane, heneicosane, tetracosane, triacontane.

In some embodiments, the lipid core also comprises a liquid lipid (e.g., tocopherol, unsaturated hydrocarbons). The ratio of solid to liquid lipids may vary from 90:10 to 50:50. For example, the ratio of solid to liquid lipids may be 90:10, 80:20, 70:30, 60:40, or 50:50.

In some embodiments, the lipid core further comprises a water insoluble alcohol (e.g., decanol, menthol).

In some embodiments, the lipid core is surrounded by a surfactant layer comprising at least one non-ionic surfactant. In some embodiments, the surfactant layer comprises a single non-ionic surfactant. In some embodiments, the surfactant layer comprises two or more non-ionic surfactants. In embodiments where a mixture of non-ionic surfactants is present, the mixture can be present in various ratios. For example, when two different non-ionic surfactants are present they may be in a ratio ranging from about 25:75 to 75:25 (w/w). In some embodiments, the non-ionic surfactants are present in a ratio from about 25:75, about 30:70, about 40:60, about 50:50, about 60:40, about 70:30, or about 75:25.

Non-ionic surfactants include those surfactants that have polar head groups that are not electrically charged. Exemplary non-ionic surfactants include alkylphenyl ethers of poly(ethylene glycol), alkylethers of poly(ethylene glycol), alkylethers of poly(propylene glycol), alkylethers of glycerol, ethanolamine derivatives, and the like. In some embodiments, the at least one non-ionic surfactant comprises a polyethylene glycol glyceride, a PEG ester, or a combination thereof. Such non-ionic surfactants include lauroyl macrogol-32 glyceride (Gelucire 44/14), PEG-32 mono- and di-esters of stearic and palmitic acids (Gelucire 48/16), and stearoyl macrogol glyceride (Gelucire 50/13).

In some embodiments, the surfactant layer further comprises an anionic surfactant. The surfactant layer may comprise more than one anionic surfactant. The ratio of the non-ionic to anionic surfactant may vary. In some embodiments, the anionic surfactant comprises about 10% to about 25% of the surfactant layer (e.g., about 10% to about 15%, about 10% to about 20%, about 15% to about 25%, about 15% to about 20% or about 20% to about 25%).

Anionic surfactants carry a negatively charged head group and include, for example, sulfates, sulfonates, phosphates, and carboxylates. In some embodiments, the anionic surfactant is a glycolic acid ethoxylate ether. Glycolic acid ethoxylate ethers include, but are not limited to glycolic acid ethoxylate oleyl ether, glycolic acid ethoxylate lauryl ether, glycolic acid ethoxylate stearyl ether, glycolic acid ethoxylate hexadecyl ether, glycolic acid ethoxylated decyl ether, glycolic acid ethoxylate tetradecyl ether, and glycolic acid ethoxylate nonylphenyl ether.

In some embodiments, total lipid to surfactant ratio is about 1:05 to about 1:2 (e.g., about 1:0.5, about 1:1, about 1:1.5, and about 1:2).

In some embodiments, the lipid nanoparticle further comprises a polymer. Polymers suitable for use in the disclosed lipid nanoparticles include those with are biocompatible and non-toxic. Exemplary polymers include, but are not limited to, polylactic-co-glycolic acid (PLGA), poly(ε-caprolactone) (PCL), polyLactic Acid (PLA), polyglutamic acid (PGA), poly(3-hydroxybutyrate-co-3-hydroxyvalerate) (PHBV), and Poly(ethylene glycol) (PEG).

The polymer may be used to incorporate a polyelectrolyte coating on the lipid nanoparticle. In some embodiments, the polyelectrolyte is an anionic polyelectrolyte. The polymer may also be used to crosslink a surfactant on the surface of the particle. For example, a reactive polymer (e.g., amine-PEG) may be incorporated into the lipid nanoparticle to allow covalent bond formation with a surfactant. The polymer may be incorporated into the lipid nanoparticle to form a hybrid nanoparticle.

Lipid nanoparticles of the present invention can be prepared by using the Phase Inversion Temperature (PIT) method, although they can also be prepared using high energy methods such as ultrasonication, microfluidization, and high-pressure homogenization with similar or different components and/or reagents. The PIT method keeps the composition constant while the temperature is changed and is well-suited to the synthesis of small particles, characterized by low polydispersity. The JAK inhibitor is combined with the lipids and then surfactants, and then the mixture is co-melted and stirred (e.g., by vortexing). Purified water is added to the mixture, and heated to the phase inversion temperature until two phases, the aqueous and organic phases, are observed. Under continued stirring, cooling of the sample causes inversion of the water-in-oil emulsion to an oil-in-water emulsion; creating very small lipid droplets in the process and a transparent nanoemulsion is formed. Alternatively, a modified oil-in-water single emulsion and solvent evaporation method can be used, e.g., for hybrid nanoparticles. Two solutions are prepared—an organic solution comprising the polymer and the liquid lipid in a solvent and an aqueous solution comprising the surfactant. The organic phase is slowly poured into the aqueous phase while being sonicated. The resulting organic solvent is removed by evaporation.

### b) Peptide Hydrogel

The hydrogel composition may be a peptide hydrogel. A peptide hydrogel is colloid gel including an internal phase and a dispersion medium, in which an aqueous solution is the dispersion medium and a self-assembled network of peptides is the internal phase. The peptides in the hydrogel are self-assembled and are folded into an β-hairpin conformation in the fibrillar network that forms the internal phase of the hydrogel. Peptide hydrogels include a sufficient elastic modulus or stiffness that allows them to maintain shape. In some embodiments, the peptides have a net positive charge at neutral pH.

Peptide hydrogels disclosed herein may be characterized by shear-thinning/recovery rheological properties. The hydrogel undergoes a gel-sol phase transition upon application of shear stress, and a sol-gel phase transition upon removal of the shear stress. Thus, application of shear stress converts the solid-like gel into a viscous gel capable of flow, and cessation of the shear results in gel recovery.

The peptide hydrogel may be a sterile hydrogel prepared with physiological and non-toxic dispersion medium suitable for administration to a subject, e.g., for delivery of JAK inhibitors to a desired location.

The peptide hydrogels described herein are stable in the presence of the encapsulated lipid nanoparticles, for example, when encapsulating the lipid nanoparticles, the hydrogel maintains its mechanical rigidity (G′).

In some embodiments, the peptide hydrogel comprises peptides having a single glutamic acid residue in the self-assembling peptide sequence.

In some embodiments, the hydrogel is protease sensitive. In particular, the hydrogel is configured to be susceptible to degradation by rejection-associated proteases, particularly matrix metallo-proteases (MMP). Thus, in some embodiments, the hydrogels disclosed herein are stable in the presence of the lipid nanoparticles, display shear-thinning/recovery mechanical properties, and are susceptible to protease cleavage.

In some embodiments, the hydrogel comprises peptides having matrix metallo-proteases (MMP) cleavage sites, e.g., in the self-assembling peptide sequence. The MMP sites may be specific for any particular MMP or may be a broad spectrum site capable of being cleaved by multiple MMPs. In some embodiments, the MMP site corresponds to the MMP or MMPs responsible for the increase of MMP activity post-transplantation. In some embodiments, the MMP site is cleavable by MMP-1b, 3, 7, 9, 12 and/or 13.

In some embodiments, the MMP cleavage site has an amino acid sequence selected from the group consisting of ELR, PLGLFAR (SEQ ID NO: 1), PLGVR (SEQ ID NO: 2), X₁X₂X₃X₄, X₅SX₆LX₇A, and PLAL, wherein X₁ is L or I, X₂, and X₃ are independently selected from any amino acid, X₄ is a hydrophobic amino acid, X₅ is a hydrophobic amino acid, X₆ is any amino acid, and X₇ is T or L.

In some embodiments, the amino acid sequence of the peptides comprises or consists of an amino acid sequence of: Z₁KZ₂EZ₃KVKVPPTELRTKZ₄KZ₅ (SEQ ID NO: 4); Z₁KZ₂EZ₃KVKVPPLGLFARTKZ₄KZ₅ (SEQ ID NO: 5); or Z₁KZ₂EZ₃KVKVPPLGVRTKZ₄KZ₅ (SEQ ID NO: 6), wherein each of Z₁, Z₂, Z₃, Z₄, and Z₅ are independently selected from I, T, V, and norvaline. In select embodiments, the amino acid sequence of the peptides comprises or consists of an amino acid sequence of: IKTEIKVKV^{D}PPLGVRTKIKV (SEQ ID NO: 7); IKVEIKVKV^{D}PPLGVRTKIKV (SEQ ID NO: 8); (nV)KTE(nV)KVKV^{D}PPLGVRTK(nV)K(nV) (SEQ ID NO: 9); and (nV)K(nV)E(nV)KVKV^{D}PPLGVRTK(nV)K(nV) (SEQ ID NO: 10), wherein nV is norvaline.

In some embodiments, the amino acid sequence of the peptides comprises or consists of an amino acid sequence of: IKVEIKVKV^{D}PP (SEQ ID NO: 11)-Z₆-V, wherein Z₆ is a 5-10 amino acid sequence comprising a MMP cleavage site, as disclosed elsewhere herein. In select embodiments, the amino acid sequence of the peptides comprises or consists of an amino acid sequence of: IKVEIKVKV^{D}PPTKIAVLTAV (SEQ ID NO: 12); IKVEIKVKV^{D}PPLALETKIKV (SEQ ID NO: 13); and IKVEIKVKV^{D}PPVSLLTAIKV (SEQ ID NO: 14).

In some embodiments, the amino acid sequence of the peptides comprises or consists of an amino acid sequence of: IK-Z₇-V^{D}PPTEIKZ₈KIZ₉V (SEQ ID NO: 15); IK-Z₇-V^{D}PPTKIKZ₈KIZ₉V (SEQ ID NO: 16); or IK-Z₇-V^{D}PPTELRZ₈KIZ₉V (SEQ ID NO: 17), wherein Z₇ is a 5-10 amino acid sequence comprising a MMP cleavage site as disclosed elsewhere herein, Z₈ is T or V, and Z₉ is K or E. In select embodiments, the amino acid sequence of the peptides comprises or consists of an amino acid sequence of: IKIAVLVKV^{D}PPTEIKTKIKV (SEQ ID NO: 18); IKVSLLTAV^{D}PPTKIKVKIEV (SEQ ID NO: 19); and IKVKIELRV^{D}PPTELRTKIKV (SEQ ID NO: 20).

In some embodiments, the amino acid sequence of the peptides comprises or consists of an amino acid sequence of any of: SEQ ID NOs: 7-10, 12-14, 18-20, and 23-28, as shown in FIG. 11.

The peptides may comprise from about 20 to about 40 residues (e.g., about 20, about 25, about 30, about 35 or about 40).

In some embodiments, the peptides disclosed herein are acetylated at the N-terminus, and/or are amidated at the C-terminus.

The hydrogel typically contains at least 0.5 wt % of the peptide(s) in an aqueous medium. In some embodiments, the hydrogel contains 0.5-2 wt % of the peptide(s). For example, the hydrogel may contain about 0.5 wt %, about 0.75 wt %, about 1 wt %, about 1.25 wt %, about 1.5 wt %, about 1.75 wt %, or about 2 wt % of the peptide(s).

The peptide hydrogel can readily be made by preparing an aqueous solution comprising one or more of the peptides disclosed herein and altering one or more characteristics of the solution, wherein a hydrogel is formed. The characteristic altered may be any characteristic that results in formation of a hydrogel upon its alteration. Suitable examples include, but are not limited to, ionic strength, temperature, concentration of a specific ion, and pH. In some embodiments, the peptides, the first JAK inhibitor, and second JAK inhibitor are mixed in a cold aqueous solution prior to triggering gelation. In some embodiments, the hydrogel can be formed in a container. In some embodiments, the hydrogel can be formed in a delivery device (e.g., a syringe).

Any buffer system can be used to form the hydrogel except phosphate based buffer systems, as phosphate buffers are known to precipitate β-hairpin peptides. Accordingly, peptide hydrogels can be formed by, for example, adding buffer of appropriate ionic strength to an aqueous solution of unfolded peptide; drawing the resulting solution into a syringe; and allowing it to gel at 25° C. directly in the syringe.

The hydrogel compositions may further include pharmaceutically acceptable carriers. The term “pharmaceutically acceptable carrier,” as used herein, means a non-toxic, inert solid, semi-solid or liquid filler, diluent, surfactant, cyclodextrins or formulation auxiliary of any type. Remington's Pharmaceutical Sciences, by E. W. Martin, Mack Publishing Co., Easton, PA, 15th Edition, 1975, describes compositions and formulations suitable for pharmaceutical delivery of the peptide hydrogels herein disclosed.

The compositions disclosed herein facilitate regioselective administration of JAK inhibitors over two distinct time courses. The first JAK inhibitor, in crystalline form, is released locally via sustained diffusion from the hydrogel. The second JAK inhibitor is released, in some embodiments, as a result of degradation by rejection-associated proteases, in lipid nanoparticles LNp which can travel to areas surrounding the site of administration for delivery to immune cells. Recently, a number of JAK inhibitors have been found to have unwanted and serious side effects which resulted in receiving a black box warning from the FDA, usually as a result of systemic (e.g., oral) administration, including increasing the risk of major cardiovascular problems, such as heart attack or stroke, cancer (e.g., non-melanoma skin cancer), blood clots in the lungs and in deep veins, serious infections, and death. The disclosed compositions ability to confer regioselective administration facilitates a reduction in side effects, lower cellular toxicity, and ease of administration compared to current formulations while harnessing the therapeutic potential of JAK inhibitors. Devices and Systems

### a) Prefilled Delivery Device

The disclosure also provides a pre-filled delivery device comprising the compositions described herein. A pre-filled delivery device is a device which is filled with the composition prior to distribution to the end user that administers the composition.

The drug delivery device may include any device configured to allow administration or delivery of a liquid or gel composition. The drug delivery device may include, without limitation, an autoinjector, a pen, an eye/ear dropper, a dropper bottle, a syringe, a pump, or a transdermal patch or implant

In some embodiments, the device is a syringe. A pre-filled syringe typically includes a containment container forming part of a syringe body, a plunger, and either an attached hypodermic needle or such features to allow a needle to be attached by the user prior to administration.

The device may contain a single dose of the composition or multiple doses which can be administered over time or at different locations.

### b) Systems

Also within the scope of the present disclosure are systems or kits that include compositions described herein. In some embodiments, the systems or kits comprise the composition described herein and a delivery device. The drug delivery device may include any device configured to allow administration or delivery of a liquid or gel composition. The drug delivery device may include, without limitation, an autoinjector, a pen, a dermal patch, an eye/ear dropper, a dropper bottle, a syringe, a pump, a wound dressing, or a transdermal patch or implant. Descriptions of delivery devices set forth above are also applicable to the systems or kits.

Individual member components of the systems or kits may be physically packaged together or separately. The components of the systems or kits may be provided in bulk packages (e.g., multi-use packages) or single-use packages. The systems or kits can also comprise instructions for using the components. The instructions are relevant materials or methodologies pertaining to the systems or kits. The materials may include any combination of the following: background information, list of components and their availability information (purchase information, etc.), brief or detailed protocols for using the compositions, troubleshooting, references, technical support, and any other related documents. Instructions can be supplied with the systems or kits or as a separate member component, either as a paper form or an electronic form which may be supplied on computer readable memory device or downloaded from an internet website, or as recorded presentation.

It is understood that the disclosed systems or kits can be employed in connection with the disclosed methods. The systems or kits may further contain additional containers or devices for use with the methods disclosed herein.

The systems or kits provided herein are in suitable packaging. Suitable packaging includes, but is not limited to, vials, bottles, jars, flexible packaging, and the like.

### 3. METHODS

### a) Inhibiting Transplant Rejection

Further provided is a method of inhibiting transplant rejection in a subject who has received an organ or tissue transplant. In some embodiments, the method includes administering a composition disclosed herein at or adjacent to/surrounding the site of the transplant, for example by injection using a delivery device loaded with the hydrogel.

In some embodiments, the transplant is an allograft. In some embodiments, the allograft is a solid organ. In select embodiments, the allograft is a vascularized composite allograft (VCA). Vascularized Composite Allografts (VCAs) involve the transplantation of multiple structures or tissues that may include skin, bone, muscles, tendon, blood vessels, nerves, and connective tissue, as a functional unit. The most common types of VCAs are for hand and face transplants. In some embodiments, when the allograft is a VCA, the hydrogel composition is contained within the VCA.

By placing the compositions described herein in or near the transplant or graph, the JAKi will be released regioselectively in two distinct time regimes. First, JAKi is released locally to transplanted tissue with immediate sustained diffusion from the hydrogel. Second, the material will release JAKi-LNp in response to localized enzymatic activity driven by the intensity of the rejection response. JAKi-LNp will deliver their cargo selectively to immune cells in the lymphoid tissues draining the graft. This design will render a regulated and localized synergism with systemic CTLA4-Ig.

### b) Treating a Disease or Disorder

The disclosure also provides methods for treating or preventing a disease or disorder comprising administration of the compositions disclosed herein. The disease or disorder may include any disease or disorder known to be at least partially mediated by a JAK protein family member.

The disease or disorder may be an autoimmune disease or an inflammatory disease.

In some embodiments, the disease or disorder is an inflammatory disease or disorder. Inflammatory diseases are characterized by activation of the immune system in a tissue or an organ to abnormal levels that may lead to abnormal function and/or disease in the tissue or organ. The inflammatory diseases and disorders that may be treated by the methods of the present invention include, but are not limited to, arthritis, rheumatoid arthritis, asthma, inflammatory bowel disease (Crohn's disease or ulcerative colitis), chronic obstructive pulmonary disease (COPD), allergic rhinitis, vasculitis (polyarteritis nodosa, temporal arteritis, Wegener's granulomatosis, Takayasu's arteritis, or Behcet's syndrome), inflammatory neuropathy, psoriasis, systemic lupus erythematosus (SLE), chronic thyroiditis, Hashimoto's thyroiditis, Addison's disease, polymyalgia rheumatica, Sjogren's syndrome, or Churg-Strauss syndrome.

In some embodiments, the disease or disorder is an autoimmune disease or disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis.

Some autoimmune disorders are also associated with an inflammatory condition. Examples of inflammatory disorders which are also autoimmune disorders that can be prevented, treated or managed in accordance with the methods of the invention include, but are not limited to, asthma, encephalitis, inflammatory bowel disease, chronic obstructive pulmonary disease (COPD), allergic disorders, pulmonary fibrosis, undifferentiated spondyloarthropathy, undifferentiated arthropathy, arthritis, inflammatory osteolysis, and chronic inflammation resulting from chronic viral or bacterial infections. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis.

The amount of the compositions of the present disclosure required for use in treatment or prevention will vary not only with the particular compound selected but also with the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of the compositions described herein can be determined by comparing their in vitro activity, and in vivo activity in animal models.

Dosage amount and interval may be adjusted individually to provide levels of the active moiety which are sufficient to maintain the modulating effects, or minimal effective concentration (MEC). The MEC will vary for each compound but can be estimated from in vivo and/or in vitro data. It should be noted that the attending physician would know how to and when to terminate, interrupt, or adjust administration due to toxicity or dysfunctions in organs or tissues. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

A wide range of second therapies may be used in conjunction with the methods of the present disclosure. The second therapy may be administration of an additional therapeutic agent or may be a second therapy not connected to administration of another agent.

The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months.

The compositions disclosed herein may be administered alone or in combination with a therapeutically effective amount of at least one additional therapeutic agent. The at least one additional therapeutic agent can be administered locally or systemically.

The at least one additional therapeutic agent may comprise immunosuppressants (e.g., azathioprine, mercaptopurine, cyclosporine, tacrolimus, and methotrexate), anti-inflammatory agents (e.g., corticosteroids and aminosalicylates), immunotherapies, chemotherapeutics, antibiotics, and analgesics.

In some embodiments, the second therapy includes immunotherapy or an immunosuppressant. Immunotherapies include chimeric antigen receptor (CAR) T-cell or T-cell transfer therapies, cytokine therapy, immunomodulators, cancer vaccines, or administration of antibodies (e.g., monoclonal antibodies). Appropriate immunosuppressive agent or agents can be selected by a skilled practitioner based on several factors, including the type of organ or tissue allograft (or the type of autoimmune disorder), the organs and tissues effected, and the general health of the subject.

In some embodiments, the subject is further administered cytotoxic T-lymphocyte-associated protein 4 (CTLA4)-Ig (such as Abatacept or Belatacept). In some embodiments, CTLA4-Ig is administered systemically.

### 4. EXAMPLES

### Example 1

Systemic Jak Inhibition Synergizes with CTLA4-Ig to Prolong Transplant Survival

The combination of CTLA4-Ig with a short-course of Tofa administration (POD-1 to POD6, twice daily via oral gavage) in a full MHC mismatch (C57BL/6 to BALB/c) mouse heart transplantation model promoted long-term graft survival (FIG. 2). Importantly, this improvement in survival was unaffected even in mice receiving hearts kept ischemic for 4h before transplantation, a clinically relevant scenario, where CTLA4-Ig efficacy is undermined by early accumulation of inflammatory mediators. Analysis of the mechanism behind the therapeutic effect of the combination of Tofa+CTLA4-Ig revealed the accumulation of a high frequency of regulatory T cells (Treg) and inhibition of the differentiation of TH1 effector T cells. Tofa was very effective at preventing the maturation of dendritic cells, both in terms of upregulation of costimulatory molecules as well as secretion of pro-inflammatory cytokines. Interestingly, Tofa did not prevent the up-regulation of MHC-II molecules, rendering DC with high Signal 1 but very low Signals 2 (costimulatory molecules) and 3 (cytokines)—a phenotype that maximizes the impact of CTLA4-Ig on alloreactive T cells and should promote their functional inactivation (anergy). Tofa only minimally affected the suppressive activity of Treg.

### Example 2

Localized, Sustained Release of JAKi Via Injectable Hydrogel Effectively Synergizes with CTLA4-Ig to Prolong Graft Survival

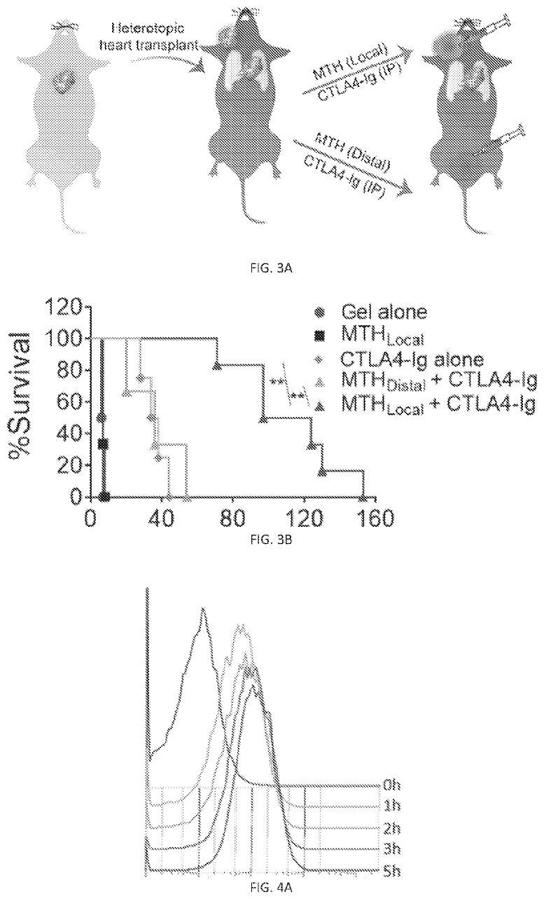

A peptide hydrogel (MAX8) that self-assembles at physiological conditions and encapsulated crystalline Tofa (cJAKi-Hydro) was used to assess that the localized release of a JAKi (Tofa) around the transplanted heart would have synergism with CTLA4-Ig as systemic Tofa administration (FIG. 3A). As shown in FIG. 3B the combination cTofa-Hydro+CTLA4-Ig induced a long-term survival in our mouse heart transplant model comparable to that obtained with systemic delivery of Tofa (that needed a total of 16 administrations). As importantly, placement of the same amount of cTofa-Hydro distal to the graft site completely ablated this synergistic protective effect. These results highlight the advantage of localized and sustained release via an injectable formulation—cJAKi-Hydro—that can exert a protective effect with a much lower dose than that conventionally required and via a single administration.

### Example 3

### Tofa-LNp Synthesis and Modulation of DC's Antigen Presenting Function

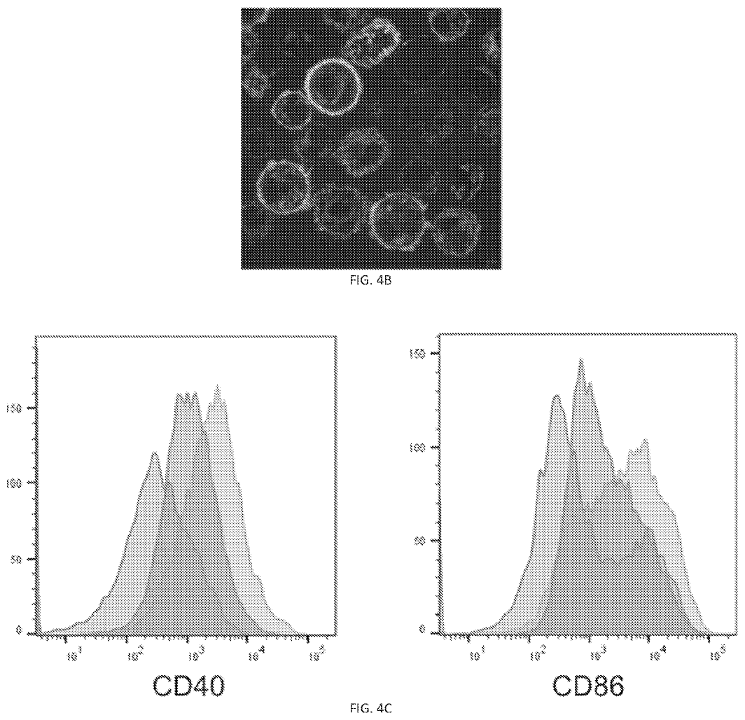

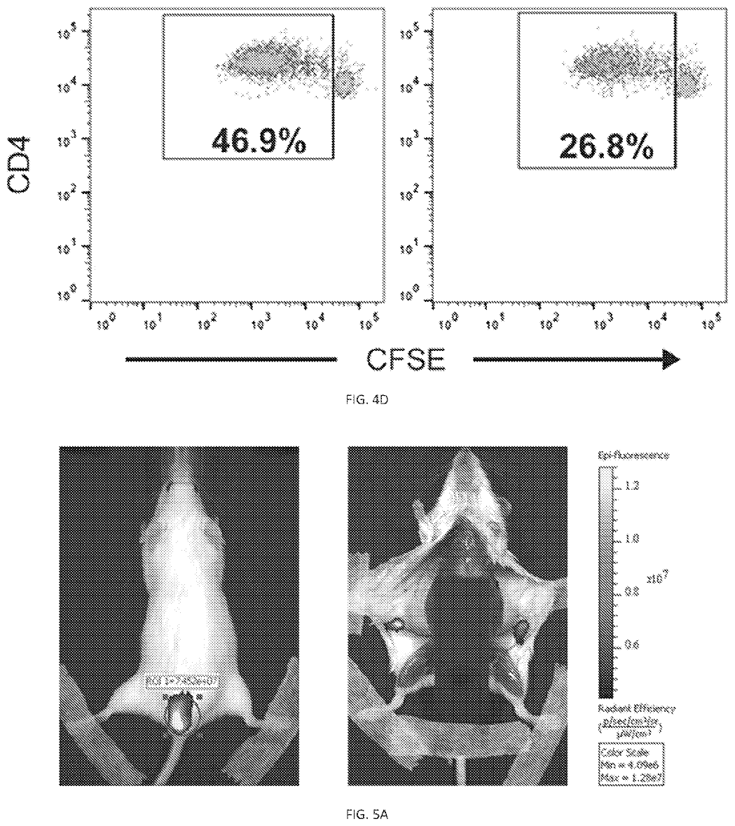

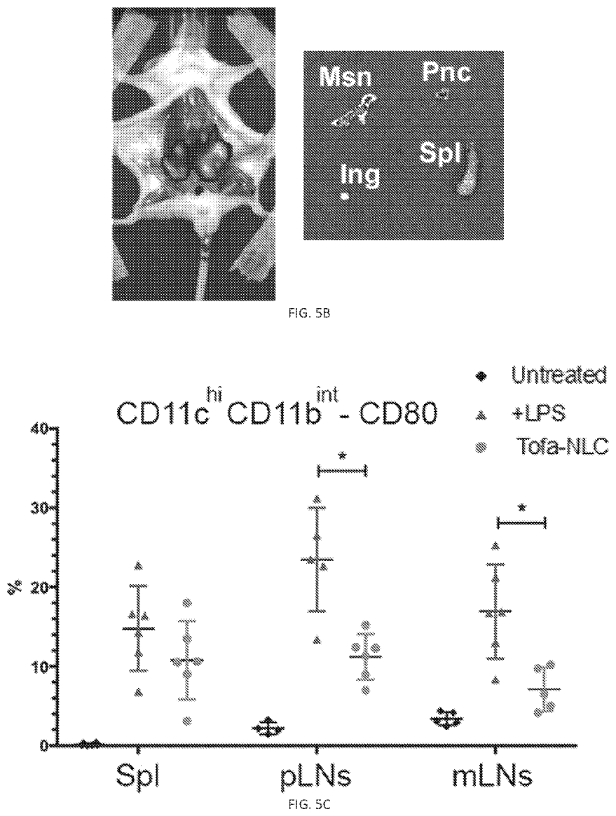

The scalable process for synthesis of ultra-small LNp was optimized using a phase inversion temperature (PIT) method. Formulations were engineered which exhibited greater than 70% encapsulation of Tofa and continuous release in vitro over approximately two days (in an infinite sink). Using fluorescent LNp, it was demonstrated that Tofa-LNp were rapidly incorporated by mouse bone marrow-derived DC, proportional to their concentration and to the length of exposure (FIGS. 4A-B). Pre-exposure to Tofa-LNp inhibited the up-regulation of maturation markers like CD40, CD80, and CD86 (FIG. 4C) to an extent comparable to that of soluble Tofa. When assessing the T cell stimulatory capacity of Empty- vs Tofa-LNp conditioned DC in a conventional CFSE-based MLR, Tofa-LNp conditioned DC were less stimulatory (FIG. 4D).

### Example 4

### LNp Biodistribution and Impact on Immune Activation

Non-invasive optical imaging was used to determine the tissue distribution of fluorescent LNp. LNp have the unique property of accumulating in lymphoid tissues, with a tropism dictated by the route of administration. For example, subcutaneous injection at the base of the tail, resulted in LNp accumulation exclusively in the inguinal lymph nodes (FIG. 5A). Following oral gavage, a strong accumulation occurred in the mesenteric lymph nodes, the pancreatic lymph nodes, and the spleen (FIG. 5B). By implementing an in vivo maturation assay, where mice are intravenously challenged with lipopolysaccharide (LPS), oral administration of Tofa-LNp was shown to affect the maturation of APCs in specific tissues (FIG. 5C). Following treatment, the maturation of CD11c^{hi}CD11b^{low} cells was blunted in both pancreatic and mesenteric lymph nodes but not in the spleen. These results confirmed that LNp are effective at delivering JAKi to draining lymphoid tissues and limit the activation of the immune system.

### Example 5

### LNp can be Encapsulated in Peptide Hydrogels and Functionally Released

The feasibility of encapsulating LNp into peptide hydrogels was tested. Fluorescent-LNp were assembled into two model peptide hydrogels (MAX8 and HLT2) and their release and integrity was assessed over time. Initial measurements indicated a quick release of LNp incompatible with the needed retention at the site of injection.

(MAX8; SEQ ID NO: 21)
VKVKVKVKV^{D}PPTKVEVKVKV
(HLT2; SEQ ID NO: 22)
VLTKVKTKV^{D}PPTKVEVKVLV

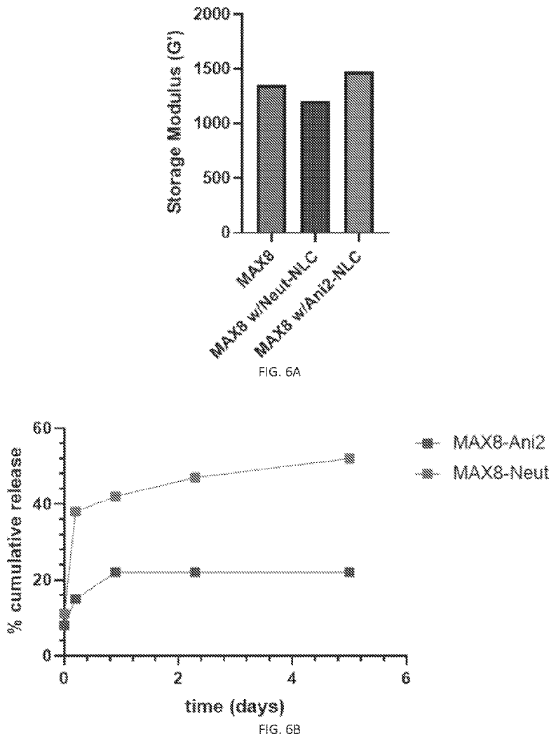

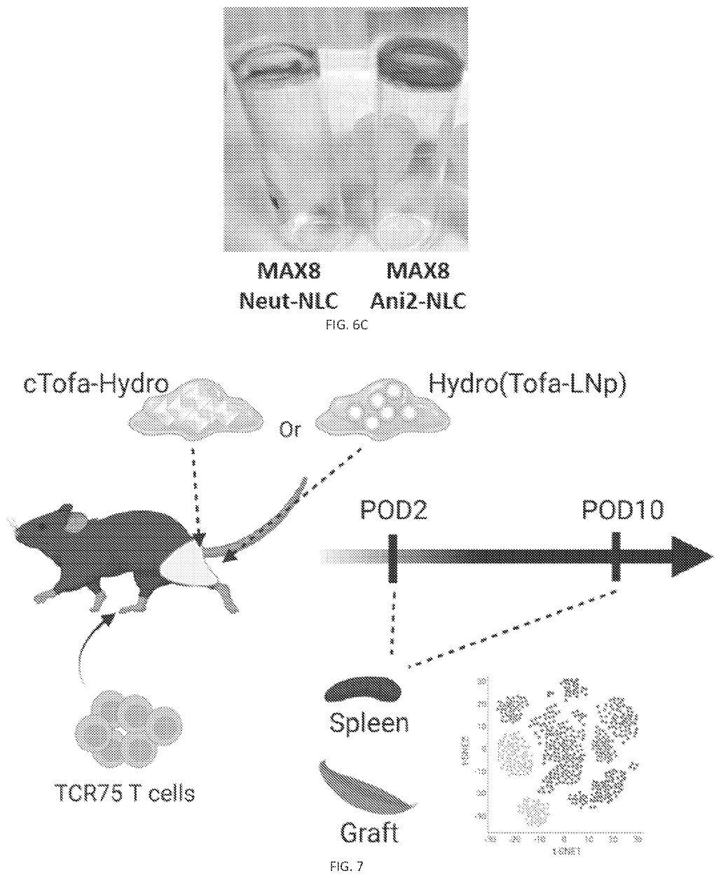

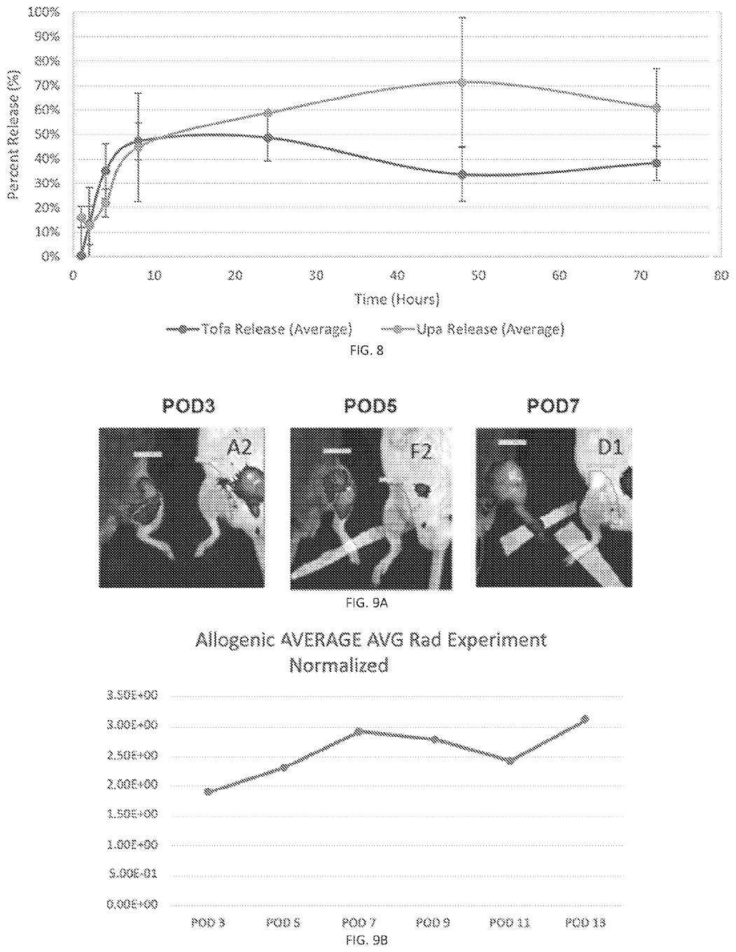

To improve particle retention within the gel, the nanoparticles were modified to achieve a slightly negative surface charge (the hydrogel network is positively charged) by adding a small percentage of anionic surfactant (Glycolic Acid Ethoxylate Oleyl Ether) together with the Gelucire 44/14. Two different ratios of these surfactants were used until similarly sized nanoparticles were achieved. FIG. 6 shows that the parent MAX8 gel stiffness is maintained following the encapsulation of either neutral or negatively-charged NLCs. FIGS. 6B-6C show that the negatively charged particles are better retained in the MAX8 gel as opposed to the neutral particles making them suitable for the formulation of use in a combination system in which a hydrogel would contain both crystallized JAKi and JAK inhibitor encapsulated in LNp,a [cJAKi-Hydro(JAKi-LNp)] delivery system.

### Example 6

### Tissue Specific Regulation of Alloreactivity by Tofa-LNp and Hydrogel Composites

Results in a heart transplant model indicated that the local release of Tofa (via a one-time implantation of hydrogel) was as effective at synergizing with systemic CTLA4-Ig as 16 oral gavage administrations (delivering 6 times more drug).

cTofa-Hydro, Hydro(Tofa-LNp) and cTofa-Hydro(Tofa-LNp) with CTLA4-Ig will be used to assess localized synergism for promoting VCA survival and the effects when using ischemic grafts. An established mouse orthotopic hind-limb transplant model in the BALB/c to C57BL/c combination, the impact on graft survival of the combination of systemic CTLA44-Ig (500 μg on PODO, 2, 4, and 6) with a one-time administration (via three subcutaneous injections, 30-50 μl each) of cTofa-Hydro, Hydro(Tofa-LNp), or cTofa-Hydro(Tofa-LNp) will be assessed using an established scale.

Additionally, the impact of the three delivery strategies on alloreactivity will be assessed using time points extrapolated from the survival results (e.g., POD10 and POD30). T cells alloreactivity will be measured via CFSE-MLR and ELISPOT and the amount of anti-donor antibodies circulating in the serum will be determined via established flow cytometry assay. The presence and activation state of infiltrating T cell subsets and dendritic cells and macrophages (high parameter flow cytometry) will be quantified alongside a pathological examination for signs of toxicity induced by our construct.

scRNAseq will be used to perform unbiased analysis of the differential regional impact of microcrystalline Tofa versus Tofa-LNp on immune cells.

To aid in the investigation of alloreactive T cells, 5×10⁵ purified congenic TCR75 Tg T cells (indirectly reactive to an I-Ad derived antigen in the context of I-Ab) will be injected the day before hind limb transplantation. As depicted in FIG. 7, recipients will be euthanized on POD2 and POD7, for evaluation of short-term and long-term effect of JAKi delivery respectively.

### Example 7

cJAKi-Hydro(JAKi-LNp) Composite Material and Control of Alloreactivity

Tofa is a pan-JAK inhibitor that reduces the kinases activity of all three JAK proteins as a function of its concentration. Multiple JAKi have been developed that possess increased selectivity (Jak1 and Jak2 in particular). To assess if a JAKi could recapitulate (or even improve upon) the effect obtained with Tofa two candidates were selected: upadacitinib (Upa, Jak1 selective) and decernotinib (Dec, Jak3 selective) for testing in functional assays, including for example, CFSE-MLR (+/−CTLA4-Ig), DC maturation, and Treg Suppression Assay.

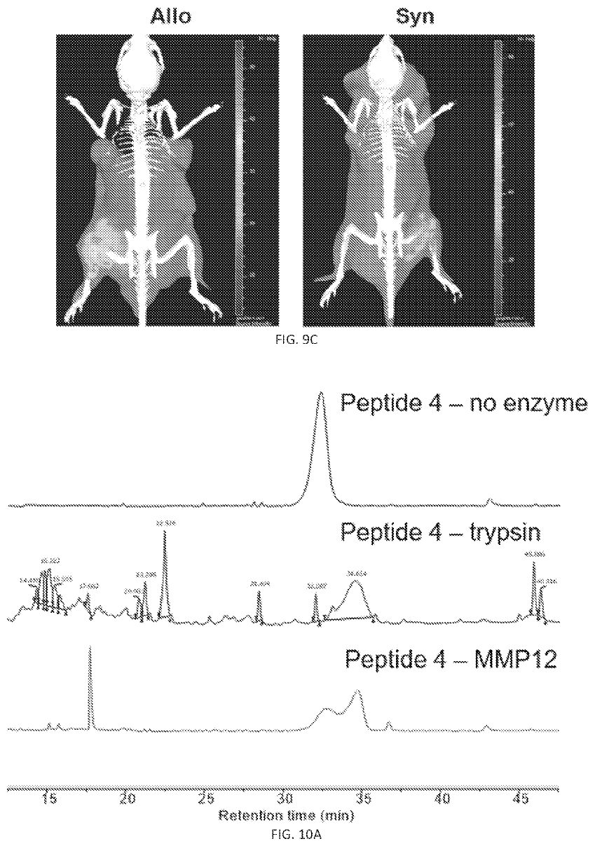

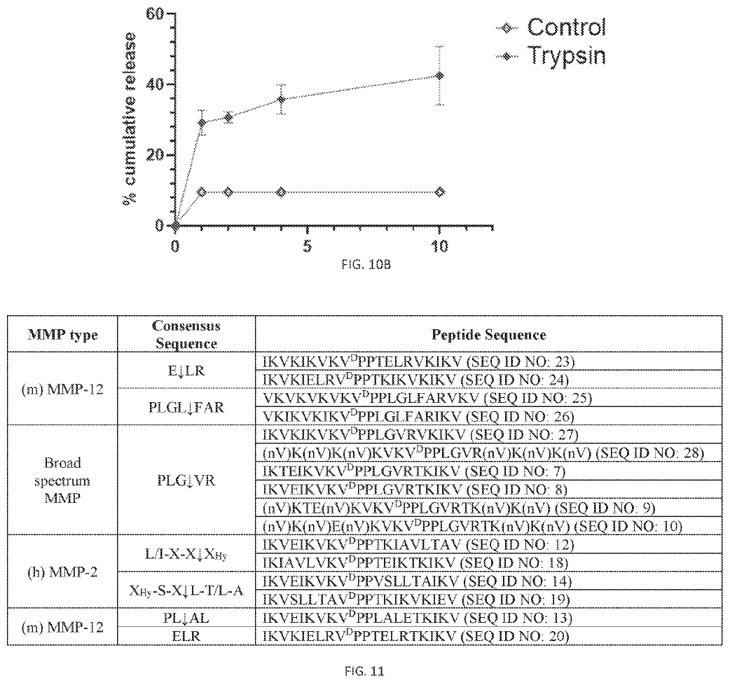

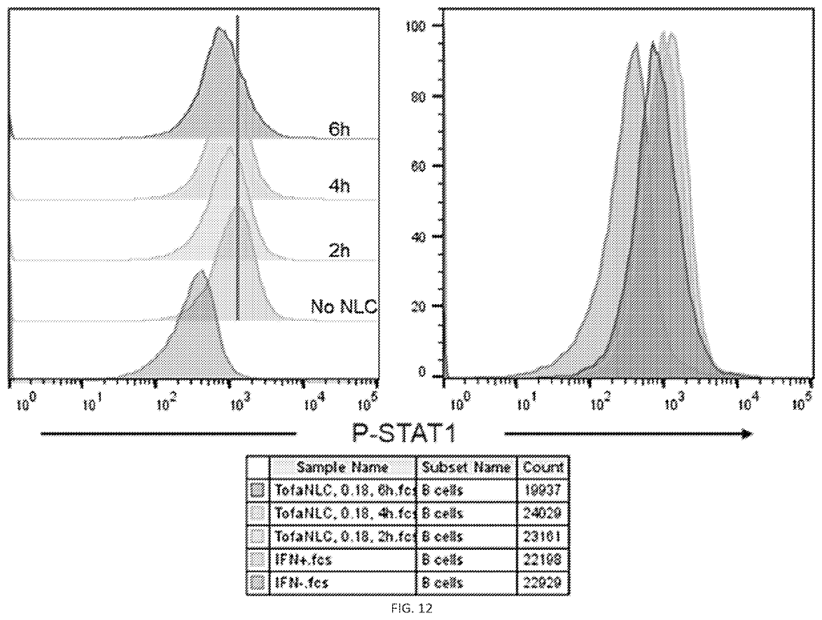

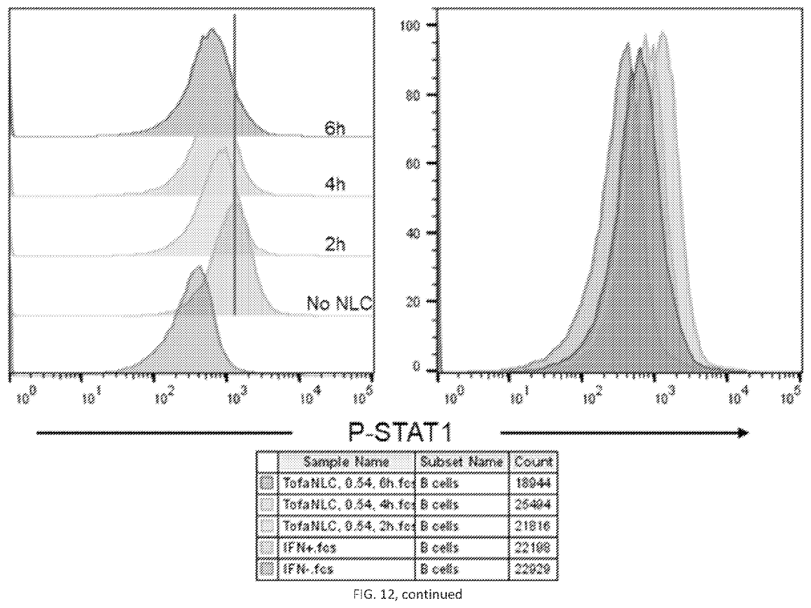

Previous LNp formulations released approximately 50% Tofa in 24 hours (FIG. 8). FIG. 9 shows the in vitro drug release profiles of both Tofa-LNp and Upa-LNp, revealing very similar kinetics. Following an initial burst release from the nanoparticles, both Tofa and Upa are slowly released over the time course to reach approximately 40-60% release by 72 hours. These preliminary results indicate the extensibility of the LNp platform to incorporation and delivery of other JAKi. They also suggest retention of 60-40% of JAKi for longterm availability.

To extend the duration of JAKi release from the LNp formulation, a small percentage of a polymer may be incorporated into the nanoparticles to create hNp. A modified oil-in-water single emulsion solvent evaporation method was used to obtain a preliminary hybrid nanoparticle (hNp) formulation that maintains compatibility with the peptide hydrogel. Particle size and polydispersity will be measured using dynamic light scattering while thermal behavior will be measured via differential scanning calorimetry to further determine suitable hNp formulations. Use with Tofa and other JAKi will test the incorporation until maximal encapsulation efficiency (EE %) is reached while maintaining the stability of the nanoemulsion, therapeutic release kinetics, cell uptake and in vivo biodistribution, particularly for the hNp formulations.

### Example 8

### Protease Sensitive Hydrogels for LNp Encapsulation

Taking advantage of the Perkin Elmer “activatable probe” MMPSense (revealing broad MMP activity), the time-dependent MMP activity is being defined in a mouse hind limb transplant model (FIG. 9). The results indicate a progressive increase of MMP activity post-transplantation that peaks around POD7 (and is maintained thereafter). Interestingly, the 3D rendering showed a difference between the “rejection signature” and the physiological “healing signature” (in syngeneic animals) both in terms of intensity and localization. Quantitative real time PCR primer sets have been validated for MMP-1b, 2, 3, 7, 9, 11, 12, 13, 14 on bone-marrow derived macrophages and can be used to quantify the expression of these MMPs on POD3 and POD7.

Peptides and corresponding hydrogels susceptible to degradation by rejection-associated proteases, particularly matrix metallo-proteases (MMP), were designed. Several peptide gels were used for formulating Hydro(JAKi-LNp) but were not compatible with the NLC particles. Encapsulation of the particles within the gel matrices resulted in the physical disruption of the gel networks as measured by significant loss of their mechanical rigidity (G′). However, the inclusion of a single glutamic acid in the self-assembling peptide's sequence dramatically increased material stiffness, affording gels that are fully compatible with the NLC particles. A series of MMP-sensitive gels were designed that incorporated MMP cleavage sites within the self-assembling peptide sequence while retaining the glutamic acid residue with facilitates optimal properties for loading with two forms of JAK inhibitors, particularly the lipid nanoparticles (FIG. 11). Peptides 2-5 incorporate -(PLGVR)- (SEQ ID NO: 2), a substrate sequence recognized by a broad range of MMP enzymes. This same sequence is incorporated in the MMPSense probe used to monitor MMP activity in the hind limb transplant model. Peptide gels have been designed that could be degraded by MMP-2 and MMP-12 and additional gels can be designed with appropriate cleavage sites that are susceptible to MMP-1, -3, -7, -9, -11, -13, and -14 if needed.

FIG. 10A shows that gels prepared from peptide 4 display protease-dependent degradation. When treated with trypsin, which cleaves at each lysine in the sequence, HPLC shows major gel disruption. When treated with MMP-12, the gel is also degraded, but cleavage is more selective as expected. Importantly, FIG. 10B shows that encapsulated LNp are released in a protease-specific manner.

Using the mouse hind-limb transplant model, the impact of the MMP-sensitive cJAKi-Hydro(JAKi-LNp) on graft survival will be assessed. Animals will receive CTLA4-Ig and a one-time administration (via three subcutaneous injections, 30-50 μl each) of the MMP-sensitive cJAKi-Hydro(JAKi-LNp). Changes in T cells alloreactivity (CFSE-MLR and ELISPOT) and circulating anti-donor antibodies (flow cytometry) will be measured along with characterization of infiltrating T cells, dendritic cells, and macrophages, and pathological examination for signs of toxicity.

### Example 9

### Tofa-LNp Tropism and Drug Release Profile

Assessments of the kinetics of inhibition of cell signaling induced by the uptake of Tofa-NLC were performed. Mouse splenocytes were pre-incubated for 2, 4, or 6 hours with two doses of Tofa-NLC: a “low” dose (starting with an equivalent of 0.18 μM Tofa before cleaning with desalting columns) that should cause inhibition only when 50% of drug content is released, and a “high” dose (starting with an equivalent of 0.54 μM Tofa before cleaning with desalting columns) that should induce appreciable inhibition of JAK/STAT signaling already at short incubation period. Cells were then stimulated with IFN-β and the induction of phosphorylated STAT1 (P-STAT1) was measured as readout of signaling integrity or inhibition (FIG. 12). In the “low dose” experiment, a small reduction of P-STAT1 accumulation was already apparent at 2h of pre-incubation with cleaned Tofa-NLC, but became much more appreciable at the 6h pre-incubation time (FIG. 12, upper panels). In the “high dose” experiment, a clear correlation between pre-incubation and degree of inhibition of P-STAT1 accumulation became apparent (FIG. 12, lower panels). These results demonstrate a progressive release of Tofa by NLC taken up by cells, resulting in an increasing degree of inhibition of the JAK/STAT signaling pathway.

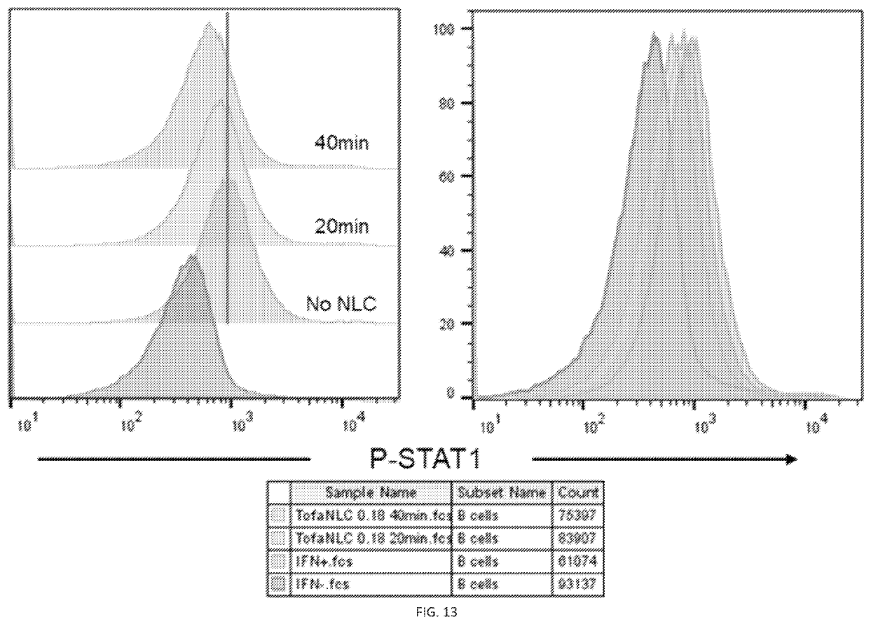

A similar experiment using shorter pre-incubation times (20 min, 40 min, 1h, 2h) of splenocytes with low and high doses of Tofa-NLC was performed. All time points for the high dose pre-incubation group presented the same degree of inhibition of P-STAT1 accumulation (not shown). Interestingly, in the low dose group, an increase in inhibition between 20 and 40 minutes of pre-incubation with Tofa-NLC was observed (FIG. 13), suggesting an interaction between immune cells and Tofa-NLC that correlates with the drug release profile measured for free particles.

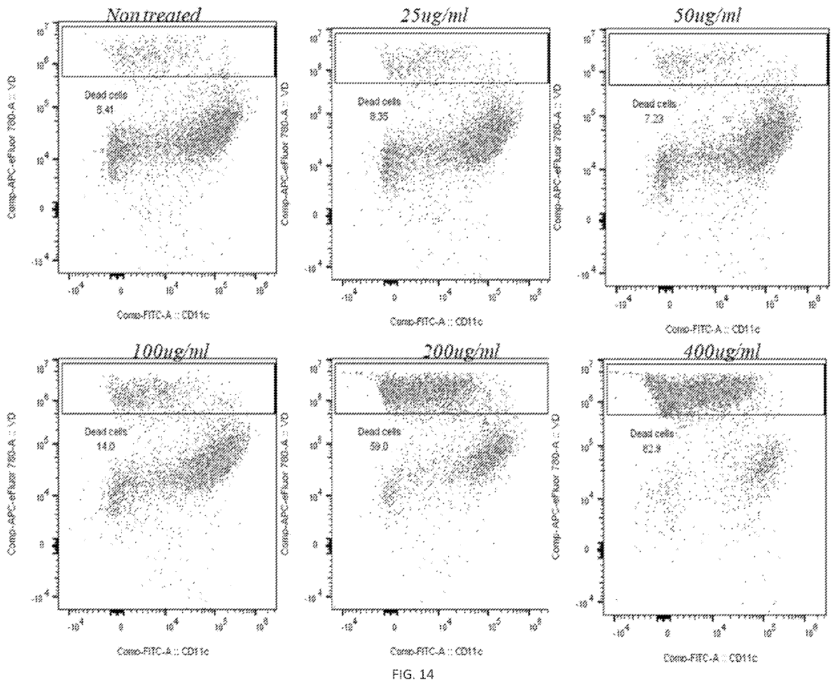

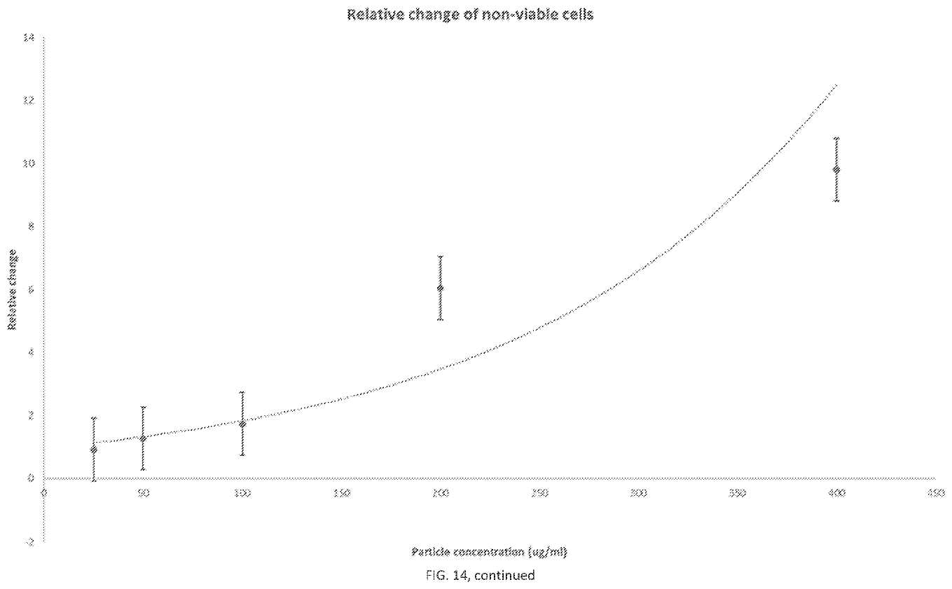

Assessment of biocompatibility of the Ani2-LNp formulation was performed. Mouse bone marrow derived dendritic cells were co-cultured overnight with a titration of Ani2-LNp and the proportion of non-viable cells assessed for each condition via flow cytometry. The first set of experiments were a “high dose” titration (2-fold dilution) starting at 400 μg/ml to 25 μg/ml of particles. Each concentration of particles was incubated with 1 million BMDCs for 18 hours. The BMDCs were then stained with a dendritic cell marker and a viability dye for flow cytometry analysis (FIG. 14). Significant cell death was seen only at a particle concentration of 100 μg/ml or higher. These results confirm the broad range of concentrations that Ani2-LNp can be used at before exerting negative effects.

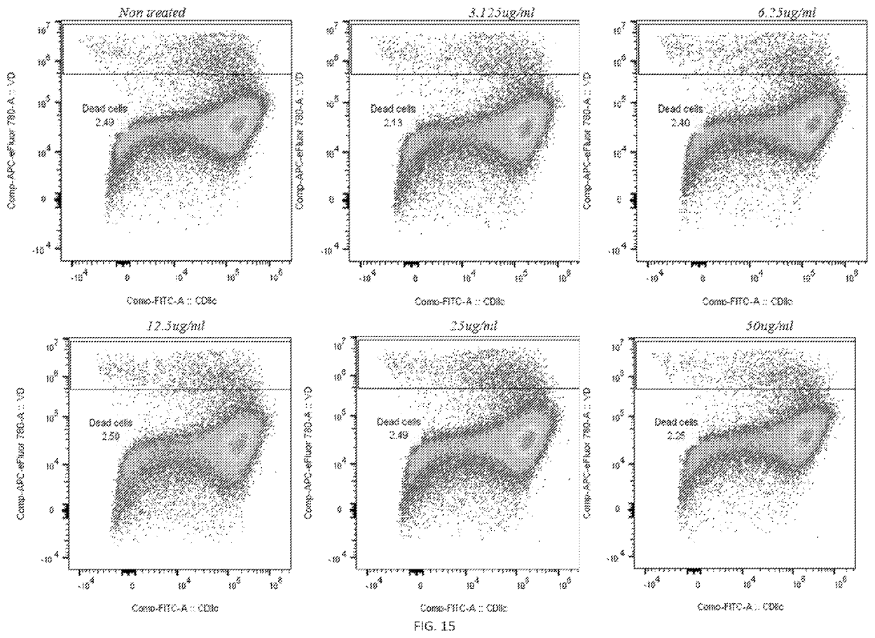

Sequential experiments used a low dose titration of the particles with the same protocol as the high dose titration experiments (FIG. 15). Because significant cell death was seen after the 100 μg/ml concentration of particles, the two-fold dilution of particles was started at 50 μg/ml. No significant cell death was seen between the control samples and experimental samples of differential low dose titration particle concentrations.

### Example 10

### In Vivo Targeted Drug Release by Hydro(Tofa-LNp) Composite

Preliminary experiments were conducted on the proteolytic stability of the peptides and the impact on the release of the encapsulated NLCs. To investigate protease-mediated release, comparative release rates of Ani2-NLCs were studied with respect to nVET2 and IVET1 gels in trypsin-rich and MMP12-rich environments. Ani2-NLCs are retained within the gels over extended periods of time due to their negative zeta potential that allows them to bind to the positively charged gel network.

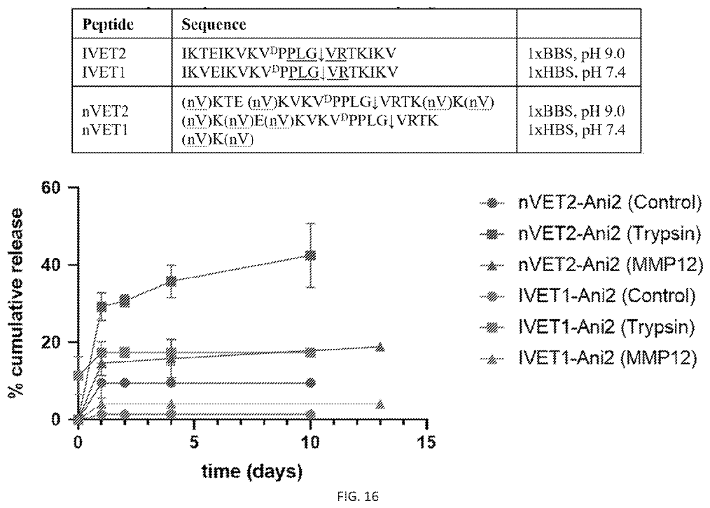

Upon trypsinization, both IVET1 and nVET2 gels show faster release rates of Ani2-NLCs than those observed in the absence of the protease. The IVET1 gels showed a nearly 12-fold increase in release rate with 17.4% of Ani2-NLCs released within the first 24 hours, relative to the 1.5% release of Ani2-NLCs in the absence of trypsin (FIG. 16). Similarly, the nVET2 gels showed a 3-fold increase in release rate with 30% release of Ani2-NLCs in the first day relative to the 10% release of Ani2-NLCs from the control gel over the same period. In the case of the MMP12 treated gels, both IVET1 and nVET2 exhibited release rates that are intermediate between their respective trypsin-rich and protease-free gel environments. These relative release trends of IVET1 and nVET2 gels are somewhat consistent with the degradation of the individual peptides by trypsin and MMP12. Trypsin hydrolyzes proteins at the carboxyl side of lysine and arginine amino acid residues and as such, the peptides IVET1 and nVET2 contain six cleavable sites with respect to trypsin. On the other hand, MMP12 recognizes a single cleavage site (PLG↓VR) within each of the peptide sequences. Thus, the observed rate of release of the NLCs is proportional to the number of protease-specific cleavage sites within the peptide sequence

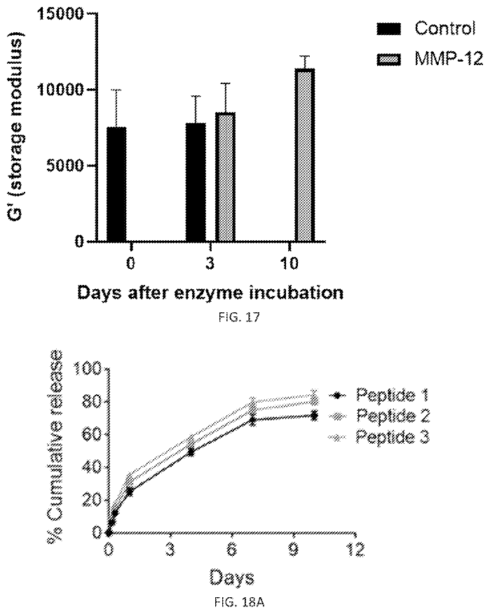

To provide further insight into the mechanistic effects of the gel release rates described above, the rheological properties of the MMP12-treated peptide gels were investigated. For this study, pre-formed IVET1 gels (150 ul, 1 wt %) were incubated at 37° C. with MMP12 (1×HBS, pH 7.4) and the storage modulus at 6.3 rad/s (obtained from frequency sweep measurements) was plotted at different time points for each gel. The gels displayed a relatively stable storage modulus over the course of 12 days with only a marginal increase in storage modulus for the MMP-treated gels, which was not statistically significant (FIG. 17). Despite peptide cleavage, the gels remained intact due to the stable cross-linked fiber network supported by the inter-hairpin hydrophobic contacts of the peptides upon self-assembly. This further suggests that MMP-programmable release of NLCs can be achieved without physical disintegration of the gel. nVET1, a nor-valine peptide analog that assembles at pH 7.4 was designed and synthesized (FIG. 16), thus avoiding the nVET2 gel system that requires initial assembly at pH 9.0 followed by additional buffer re-equilibration at neutral pH prior to any further application.

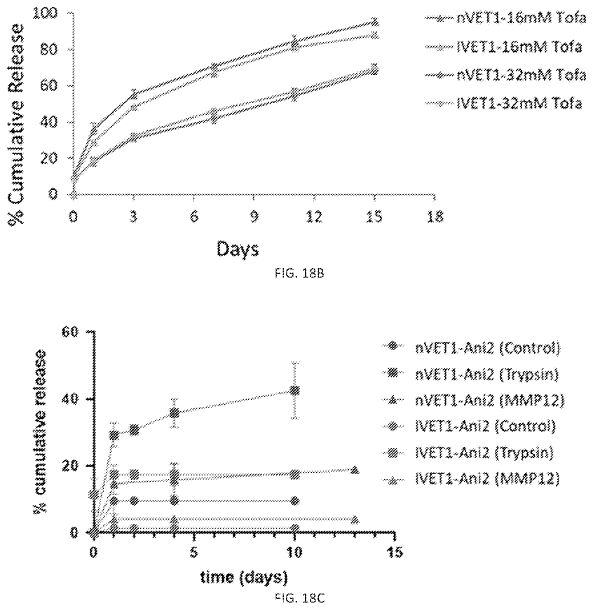

The release rates of microcrystalline tofacitinib from select peptide gels (MAX8 and HLT2) was determined as in Majumder, P., et al., Small 2020, 16, 2002791, incorporated herein by reference (FIG. 18A). As such, the release rates were established for the enzyme cleavable peptides (nVET1 and IVET1) at two different drug loads—16 mM and 32 mM. The release of tofacitinib was quantified using UV-Vis spectroscopy (λ=285 nm). The release rates of Tofa (FIG. 18B) from the enzyme cleavable nVET1 and IVET1 gels (charge=+5) containing 16 mM microcrystalline Tofacitinib, are comparable with those reported for peptide gels MAX8 (Peptide 1, +7), MAXI (Peptide 2, +9) and HLT2 (Peptide 3, +5) (FIG. 18A). All 5 peptide gels demonstrated a sustained release of payload over a week with 70-80% of tofacitinib released after 10 days. Further, increasing the concentration of microcrystalline deposits to 32 mM resulted in an even slower release profile for the nVET1 and IVET1 gels (FIG. 18B), with less than 60% of drug release after 10 days. The release profile is thus dependent on the final concentration of drug loaded and consistent with crystal dissolution being rate-determining. In addition, the close overlap in release curves between the nVET1 and IVET1 peptide gels, for a given concentration of drug loading, suggests that the Tofa release is independent of the nature of the peptide hydrogel.

The release rates of Ani2-NLCs from the nVET1 and IVET1 gels (FIG. 18C) were significantly slower than the diffusion-based release rate of crystalline Tofa in the absence of enzymatic action. At the end of 10 days, less than 20% of Ani2-NLCs were released in the absence of enzyme. Thus, two distinct release regimes of tofacitinib are attainable with simultaneous incorporation of the drug as microcrystalline deposits and as Tofa-NLCs.

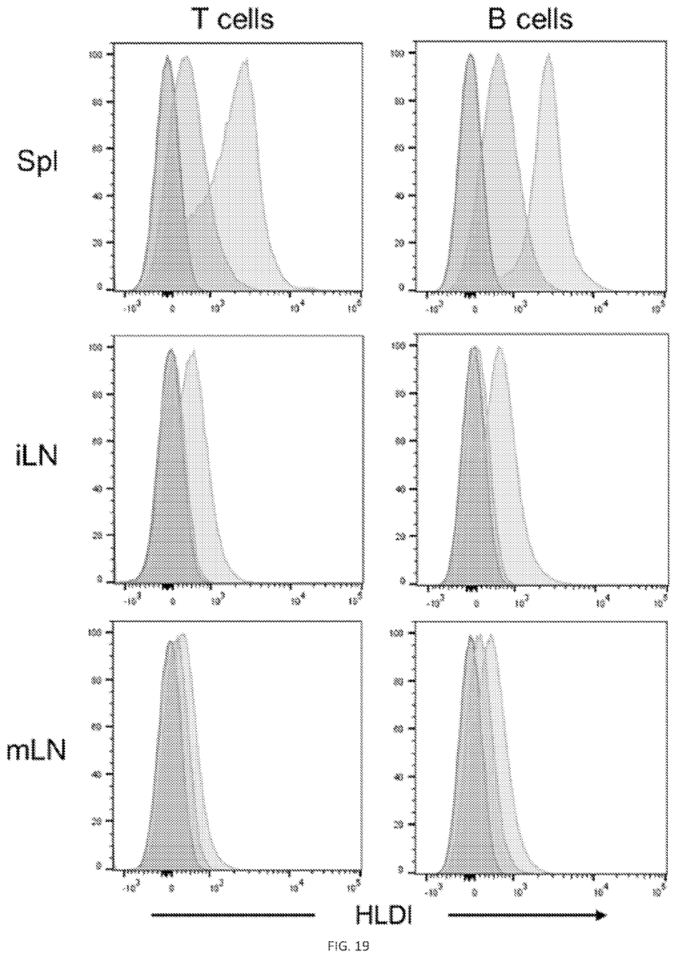

In vivo LNp biodistribution The Tofa in the particles was replaced with HLDI fluorescent molecules. Following subcutaneous injection of HLDI-NLC in the dorsal area near the base of the tail, the distribution and uptake was monitored. In this first experiment, the mice were euthanized at 1 h and 2h post injection. The spleen resulted the tissue of highest fluorescence accumulation (FIG. 19), with strongest intensity at 2 h and comparable uptake by all immune subsets interrogated (Macrophages, Dendritic Cells, T cells, B cells). The inguinal lymph nodes presented a significant intensity of fluorescence at 2h. Given that both T cells and B cells presented comparable levels of HDLI signal suggests that the particles travelled autonomously to the inguinal lymph nodes rather than being carried by cells (like dendritic cells or macrophages) sampling the site of injection. Mesenteric lymph nodes immune cells were only minimally fluorescent at both time point analyzed, confirming the differential draining of the injection site used.

### Example 12

Localized Synergism of Hydro(Tofa-LNp) with CTLA4-Ig to Promote Mouse Hind Limb Survival

To initiate testing the impact in the mouse model of hind limb transplantation, the lack of toxicity of our hydrogel (Max8) following subcutaneous (SQ) injection was confirmed. To this end, the condition of transplants was replicated by surgically separating the skin of the prospective area of injection (flap raise) followed by suturing the edges. After completion of the surgical procedure, empty hydrogel or Tofa containing hydrogel was injected SQ and the area monitored visually for any macroscopic sign of toxicity. The results (FIG. 20) show absence of any evidence of toxicity associated with application of hydrogel.

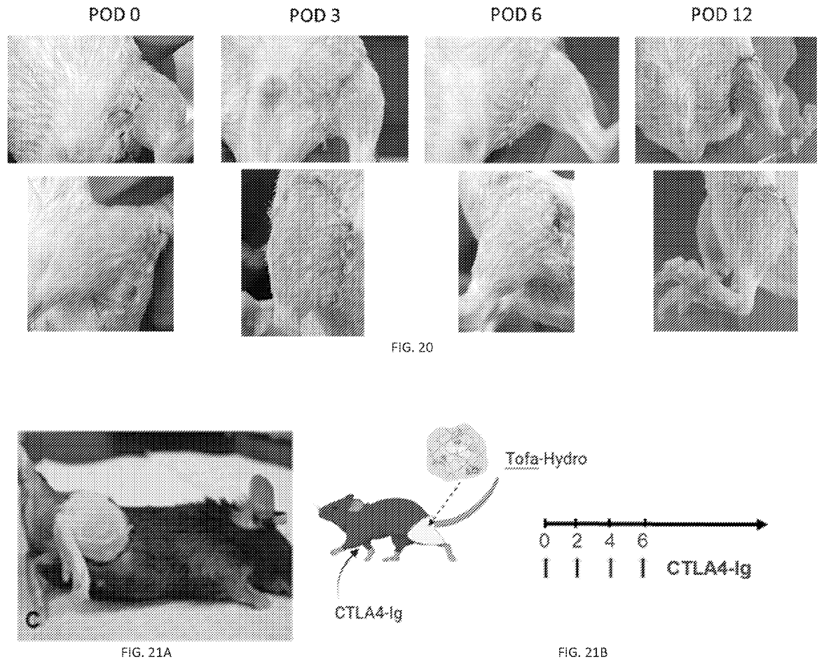

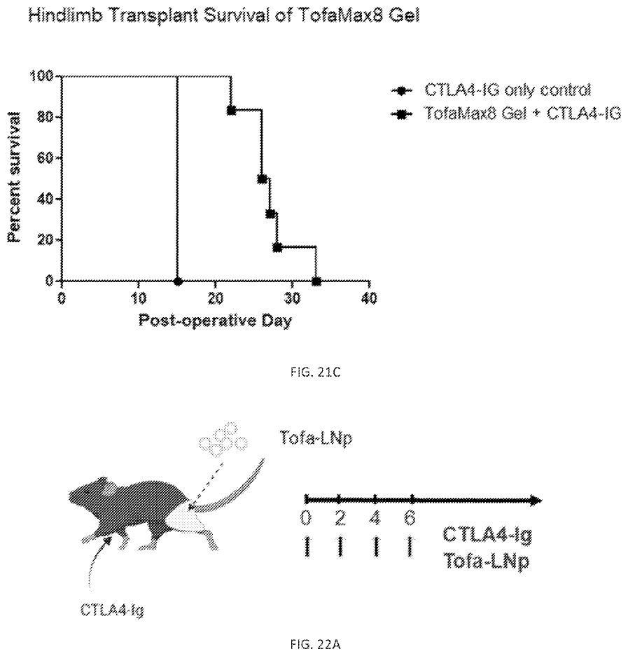

The impact of local injection hydrogel (Max8) containing crystalline Tofa (See Majumder, P., et al.) in the mouse model of orthotopic hind limb transplantation (FIGS. 21A-21B) was examined. In comparison to the modest extension of transplant survival promoted by the systemic administration of the biologic CTLA4-Ig, a single application of Tofa-Hydrogel at the time of transplant synergized with CTLA4-Ig to induce a significant extension of graft survival (FIG. 21C).

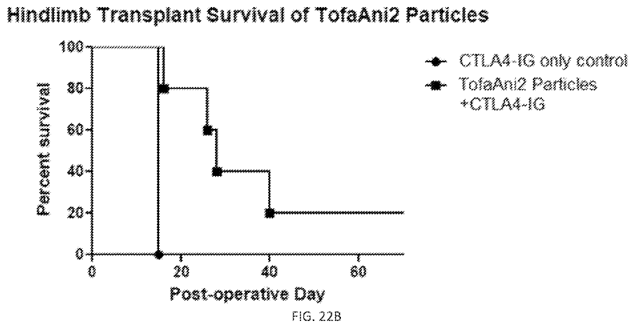

Similarly, the impact of multiple local administrations of Tofa-Ani2 was examined following a regimen that would simulate the progressive release of particles from the hydrogel (FIG. 22A). Four every other day injections of Tofa-Ani2 local to the transplant synergized with CTLA4-Ig administration and promoted a significant extension of graft survival (FIG. 22B).

Each component of the proposed LNp-hydrogel composite exerted a significant therapeutic effect in the context of transplant rejection.

### Example 13

### Impact of Hydrogel Composites on Host Ability to Resolve Infection

A localized immunomodulatory strategy avoids drug related systemic side effects while preserving the functionality of the host immune system at distal sites. The impact of three hydrogel composite formulations on the resolution of S. aureus infection, a type of infection that transplant patients are more susceptible to, will be studies. Luciferase-expressing S. aureus is injected in the dorsal skin of mice and the resolution of infection when the composites [cTofa-Hydro, Hydro(Tofa-LNp), or cTofa-Hydro(Tofa-LNp)] is applied in the leg of the animal is monitored. The infection is commonly resolved in 14 days and any significant delay or advance in resolution will indicate an impact from the delivery strategy. A repeat of the cohorts is used to define any difference in the skin and draining LN accumulation of neutrophils, monocytes, macrophages, DC, and IL-17A and IL-22 producing T cells in response to infection.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A hydrogel composition comprising:
a first JAK inhibitor, or a pharmaceutically acceptable salt thereof; and
a second JAK inhibitor, or a pharmaceutically acceptable salt thereof, encapsulated in a lipid nanoparticle.

2. (canceled)

3. The hydrogel composition of claim 1, wherein the first JAK inhibitor, the second JAK inhibitor, or both is tofacitinib, or a pharmaceutically acceptable salt thereof.

4. The hydrogel composition of claim 1, wherein the first JAK inhibitor, or a pharmaceutically acceptable salt thereof, is contained within microcrystalline deposits.

5. The hydrogel composition of claim 1, wherein the lipid nanoparticle comprises a lipid core comprising a saturated hydrocarbon having 19 to 30 carbons and a liquid lipid, and optionally, a polymer.

6. The hydrogel composition of claim 5, wherein the lipid core is surrounded by a surfactant layer comprising at least one non-ionic surfactant, and optionally, an anionic surfactant.

7-8. (canceled)

9. The hydrogel composition of claim 6, wherein the anionic surfactant comprises 10-15% of the surfactant layer.

10. (canceled)

11. The hydrogel composition of claim 6, wherein the lipid nanoparticle has a lipid to surfactant ratio of 1:0.5 to 1:2.

12. (canceled)

13. The hydrogel composition of claim 1, wherein the hydrogel is a peptide hydrogel.

14. The hydrogel composition of claim 1, wherein the hydrogel is protease sensitive.

15. The hydrogel composition of claim 14, wherein the hydrogel comprises peptides having at least one matrix metallo-proteases (MMP) cleavage site, wherein each MMP cleavage site comprises an amino acid sequence selected from the group consisting of ELR, PLGLFAR (SEQ ID NO: 1), PLGVR (SEQ ID NO: 2), X₁X₂X₃X₄, X₅SX₆LX₇A, and PLAL (SEQ ID NO: 3), wherein X₁ is L or I, X₂, and X₃ are independently selected from any amino acid, X₄ is a hydrophobic amino acid, X₅ is a hydrophobic amino acid, X₆ is any amino acid, and X₇ is T or L.

16. (canceled)

17. The hydrogel composition of claim 13, wherein the peptides comprise an amino acid sequence of:
(SEQ ID NO: 4)
Z₁KZ₂EZ₃KVKVPPTELRTKZ₄KZ₅;
(SEQ ID NO: 5)
Z₁KZ₂EZ₃KVKVPPLGLFARTKZ₄KZ₅;
(SEQ ID NO: 6)
Z₁KZ₂EZ₃KVKVPPLGVRTKZ₄KZ₅;
(SEQ ID NO: 11)-Z₆-V
IKVEIKVKV^{D}PP;
(SEQ ID NO: 15)
IK-Z₇-V^{D}PPTEIKZ₈KIZ₉V;
(SEQ ID NO: 16)
IK-Z₇-V^{D}PPTKIKZ₈KIZ₉V;
or
(SEQ ID NO: 17)
IK-Z₇-V^{D}PPTELRZ₈KIZ₉V,
wherein each of Z₁, Z₂, Z₃, Z₄, and Z₅ are independently selected from I, T, V, and norvaline,
Z₆ is a 5-10 amino acid sequence comprising the MMP cleavage site, and
Z₇ is a 5-10 amino acid sequence comprising the MMP cleavage site, Z₈ is T or V, and Z₉ is K or E.

18. The hydrogel composition of claim 17, wherein the peptides comprise an amino acid sequence selected from:
(SEQ ID NO: 7)
IKTEIKVKV^{D}PPLGVRTKIKV;
(SEQ ID NO: 8)
IKVEIKVKVPPPLGVRTKIKV;
(SEQ ID NO: 9)
(nV)KTE(nV)KVKVPPPLGVRTK(nV)K(nV)
and
(SEQ ID NO: 10)
(nV)K(nV)E(nV)KVKVPPPLGVRTK(nV)K(nV)
(SEQ ID NO: 12)
IKVEIKVKV^{D}PPTKIAVLTAV;
(SEQ ID NO: 13)
IKVEIKVKV^{D}PPLALETKIKV;
(SEQ ID NO: 14)
IKVEIKVKV^{D}PPVSLLTAIKV;
(SEQ ID NO: 18)
IKIAVLVKV^{D}PPTEIKTKIKV;
(SEQ ID NO: 19)
IKVSLLTAV^{D}PPTKIKVKIEV;
and
(SEQ ID NO: 20)
IKVKIELRV^{D}PPTELRTKIKV,
wherein nV is norvaline.

19-22. (canceled)

23. The hydrogel composition of claim 1, wherein the hydrogel is a shear-thinning hydrogel.

24. (canceled)

25. A pre-filled delivery device comprising the hydrogel composition of claim 1.

26-27. (canceled)

28. A method of inhibiting transplant rejection in a subject who has received an organ or tissue transplant, comprising administering the hydrogel composition of claim 1 at or adjacent to the site of the transplant.

29. The method of claim 28, wherein the transplant is an allograft.

30. (canceled)

31. The method of claim 28, wherein the allograft is a vascularized composite allograft (VCA) and the hydrogel composition is contained within the VCA.

32. (canceled)

33. A method for treating or preventing an autoimmune or an inflammatory disease or disorder disease or disorder comprising administering the hydrogel composition of claim 1 to a subject in need thereof.

34. (canceled)

35. The method of claim 28, further comprising administering cytotoxic T-lymphocyte-associated protein 4 (CTLA4)-Ig to the subject.

36. A system or kit comprising:
the hydrogel composition of claim 1; and
a delivery device.

37-38. (canceled)
